(19) United States
(12) Patent Application Publication
(10) Pub. No.: US 2025/0161371 A1
(43) Pub. Date: May 22, 2025

(54) REGENERATIVE HELMINTH CONTAINING COMPOSITIONS AND USES THEREOF

(71) Applicant: The Johns Hopkins University, Baltimore, MD (US)

(72) Inventors: Jennifer Elisseeff, Baltimore, MD (US); Drew M. Pardoll, Baltimore, MD (US); David R. Maestas, Baltimore, MD (US)

(21) Appl. No.: 18/894,513

(22) Filed: Sep. 24, 2024

Related U.S. Application Data
(63) Continuation of application No. PCT/US2023/016483, filed on Mar. 28, 2023.
(60) Provisional application No. 63/324,908, filed on Mar. 29, 2022.

(51) Int. Cl. A61K 35/62; A61K 9/06; A61P 27/02

(52) U.S. Cl. CPC A61K 35/62; A61K 9/06; A61P 27/02

(57) ABSTRACT

Disclosed herein are compositions containing a mixture of an soluble fraction and insoluble fraction derived from at least one helminth egg for use in treating wounds and/or injuries to tissues in subjects in need of treatment thereof.

### RELATED APPLICATION INFORMATION

The present application is a continuation of International Application No. PCT/US2023/016483 filed on Mar. 28, 2023, which claims priority to U.S. Application No. 63/324,908 filed on Mar. 29, 2022, the contents of which are herein incorporated by reference.

### STATEMENT OF GOVERNMENT SUPPORT

This invention was made with government support under W81XWH-19-1-0576 awarded by the Defense Health Agency, Medical Research and Development Branch, and AR076959 awarded by the National Institutes of Health. The government has certain rights in the invention.

### TECHNICAL FIELD

The present disclosure relates to regenerative compositions containing a mixture of a soluble fraction and insoluble fraction derived from one or more helminth eggs. The compositions can be used to promote wound healing and tissue repair in subjects in need of treatment thereof.

### BACKGROUND

Parasitic worm infections affect billions of people worldwide, primarily in tropical developing regions, where they can cause morbidity and mortality (6-8). One such parasite, Schistosoma mansoni (S. mansoni), is a helminth that can infect and can cause severe damage to the liver and hepatic blood vasculature due in part to the immune responses against the eggs lodged in hepatic sinusoids. These responses to xenogeneic foreign bodies and their respective secretome lead to granuloma formation, and if left unresolved can result in fibrosis (9-12). While S. mansoni and other helminth infections can cause a variety of disease states, they also appear to promote a number of desirable features. For example, some helminth infections reduce the incidence of allergies and are associated with beneficial alterations in the microbiome (13, 14). Infection has also been associated with decreased symptom severity in auto-immune disease and even Alzheimer's disease symptoms, suggesting helminths may induce beneficial immunomodulation (15-17). More directly connecting helminth responses to tissue repair, Nusse et al. demonstrated that a Heligmosonoides polygyrus infection triggers epithelial stem cell niche reprogramming, suggesting a connection between helminths and tissue repair that is modulated by the immune response to the worm and its egg secretome (18, 19). Finally, helminth infection induces IL-4 receptor (IL-4R) signaling that downregulates IL-17A production to benefit rapid tissue repair after the initial infection (20).

Type 2 immunity is characterized by the influx of T helper type 2 (TH2) cells, eosinophils, type 2 innate lymphoid cells (ILC2), alternatively activated macrophages, and type 2 associated cytokines IL-4, IL-5, IL-9, and IL-13 (18, 21). Previous studies found that the egg secretome components of helminth (e.g., such as S. mansoni) and the soluble egg antigen (SEA) can stimulate type 2 immunity, recapitulating in part the immune response to infection. Evidence suggest that multiple components of SEA can contribute to the type 2 immune responses, in particular the glycoproteins Omega-1, IPSE/alpha-1 (IL-4 inducing principle of S. mansoni eggs), and the Lewis^{X} containing glycan LNFP III (22-24).

While the type 2 immune response is historically considered a protective response against parasitic worms, new perspectives suggest that helminths may be co-opting this immune response to repair the damage induced during infection by agonizing type 2 cytokines. For example, IL-4 secreted by eosinophils and TH2 cells can promote activation of other cell types required for skeletal muscle repair (4, 5). In the central nervous system, IL-4 secreting TH2 cells promote repair in the retina and spinal cord and IL-4 levels are positively associated with learning in Alzheimer's disease animal models (25-27). Although helminths drive type 2 responses, it is unknown whether helminth secretory agents can be formulated or engineered as safe and effective regenerative immunotherapies without the pathologies coincident with parasitic infections such as fibrosis and hepatic damage (28-30).

There is a need in the art for new regenerative immunotherapies that can stimulate a type 2 immune response to promote regeneration and repair of wounds and/or tissue in subjects in need of treatment thereof.

### SUMMARY

In one aspect, the present disclosure relates to a regenerative composition comprising a mixture of a soluble fraction and an insoluble fraction derived from one or more helminth eggs, wherein the composition is produced by a process comprising the steps of:
homogenizing one or more helminth eggs;
centrifuging the homogenized helminth eggs to form an insoluble fraction, a soluble fraction, and an egg shell fraction;
removing the egg shell layer fraction;
ultracentrifuging the soluble fraction and insoluble fraction;
filtering each of the soluble and insoluble layers separately; and
combining the filtered soluble fraction with the filtered insoluble fraction in a ratio sufficient to reduce a proinflammatory response when administered to a subject, wherein the regenerative composition comprises a mixture of the soluble fraction and the insoluble fraction.

In some aspects the filtered soluble and filtered insoluble fraction is combined in a ratio of about 9:1 to about 6:1 to form a composition comprising a mixture of a soluble fraction and an insoluble fraction.

In one aspect, the helminth is Schistosoma mansoni (S. mansoni).

In another aspects, the soluble fraction comprises at least one helminth egg antigen. In yet further aspects, the at least one helminth egg antigen is one or more of omega-1, IPSE/alpha-1, secretory glycoprotein kappa-5, smp40, Histone H2A, fructose-bisphosphate aldolase, lacto-N-fucopentaose-III (LNFPIII), lacto-n-Neotetraose (LNnT), or any combinations thereof.

In other aspects, the insoluble fraction comprises at least one lipid. In yet further aspects, the at least one lipid is one or more of prostaglandin D2 (PGD2), prostaglandin E2 (PGE2), 5-hydroxyeicosatetraenoic (HETE), 15-HETE, linoleic acid, arachidonic acid, docosahexaenoic acid, 5S,18R-dihydroxy-6E,8Z,11Z,14Z,16E-eicosapentaenoic acid, 10S, 17S-dihydroxydocosa-4Z,7Z,11E,13Z,15E,19Z-hexaenoic acid, (4Z,7Z,10R,11E,13E,15Z,17S,19Z)-10,17-dihydroxydocosa-4,7,11,13,15,19-hexaenoic acid, lysophosphatidylcholine, or any combinations thereof.

In another aspect, in the above composition, the helminth eggs are at least about 90% homogenized.

In still yet another aspect, the composition further comprises an extracellular matrix. In some aspects, the composition is co-formulated with at least one extracellular matrix.

In still a further aspect, the composition is further incorporated into a gel or hydrogel. Specifically, the gel is a vitrified gel.

In still a further aspect, the above composition, when administered to a subject, increases IL-4 and decreases or reduces IL-17.

In yet another aspect, the present disclosure relates to a method of treating a wound or injury to a tissue in a subject, the method comprising the step of administering to the subject in need of treatment thereof, an effective amount of above-described composition. In some aspects, the subject is a human. In yet other aspects, the tissue with the wound and/or injury is muscle, an organ, cartilage, a ligament, skin, bone, nervous tissue, corneal tissue, a lens, or any combination thereof.

In yet a further aspect, the present disclosure relates to a kit containing the above-described composition.

### BRIEF DESCRIPTION OF THE DRAWINGS

FIG. 1 shows SEA isolation process from S. mansoni eggs. FIG. 1A shows graphical images of S. mansoni adult worms and eggs, and brightfield images of the egg disruption process. FIG. 1B shows images of the S. mansoni parasite eggs at various stages of disruption. FIG. 1C shows a graphical schematic of SEA isolation and removal of eggshell debris.

FIG. 2 shows SEA In vitro screening CD4⁺IL4⁺. FIG. 2A shows the representative flow cytometry plots of the IL-4 expression obtained in 4get splenocyte cultures. FIG. 2B shows flow cytometry populations for IL4⁺CD4⁺ cells as percentages of CD4 and CD45 live with SEA treatment versus controls. FIG. 2C shows flow cytometry population counts from treatment with IL-4 and SEA. Statistical tests represent all in vitro replicates, and all experiments were replicated at least twice. Graphs show mean±s.d. (b), n=3. * P<0.05, ** P<0.01, *** P<0.001, **** P<0.0001 by one-way ANOVA with Tukey's multiple comparisons (FIG. 2B).

FIG. 3 shows SEA in vitro Screening CD8⁺ T Cells. FIG. 3A shows representative flow cytometry plots of the CD4⁺ and CD8⁺ populations in day 5 splenocyte cultures. FIG. 3B shows flow cytometry populations for CD4⁺ and CD8⁺ cells of different dosage schemes of SEA. FIG. 3C shows flow cytometry populations for CD8⁺ cells in day 5 splenocyte cultures from an independent experiment comparing rSEA and SEA. FIG. 3D shows flow cytometry population counts from treatment with standard SEA and an alternative SEA formulation. Statistical tests represent all in vitro replicates, and all experiments were replicated at least twice. Graphs show mean±s.d. (b, c), n=3. * P<0.05, ** P<0.01, *** P<0.001, **** P<0.0001 by one-way ANOVA with Tukey's multiple comparisons (FIG. 3B, FIG. 3D) or unpaired, two-tailed T-test (FIG. 3C).

FIG. 4 shows treatment with SEA increases Il4 gene expression with a small rise in pro-inflammatory genes in local muscle injury tissue. FIG. 4A shows gene expression of Il4 and Gata3 in muscles treated with SEA, assessed 1-week post-injury. FIG. 4B shows gene expression of Illb, Tnfa, and Ifng in muscles treated with SEA, assessed 1-week post injury. Statistical tests represent all biological replicates, and all experiments were replicated at least twice. Graphs show mean±s.d. (FIG. 4A, FIG. 4B), n=4. * P<0.05, **** P<0.0001 by two-way ANOVA with Sidak's multiple comparisons.

FIG. 5 shows SEA vs rSEA compositional comparison and impact on cell proliferation. FIG. 5A shows SDS-PAGE gel to visualize changes to the SEA proteins when alternate isolation techniques are performed, using 5 μg of protein/lane. Columns 1 and 2 represent SEA and rSEA before (1) and after (2) sterile filtration. FIG. 5B shows mass chromatogram from LC-MS run showing increased lipid levels in rSEA versus SEA. FIG. 5C shows a list of the 50 most highly upregulated lipid compounds in rSEA vs SEA. FIG. 5D shows a comparison of lipid class levels; black bars indicate classes that are significantly upregulated in rSEA vs SEA. FIG. 5E shows a primary 4get splenocyte culture day 5 results on total live CD45⁺ cell number increases with SEA and rSEA at the same dosage of 20 μg in 200 μL of complete culture media. Statistical tests (FIG. 5B) represent all in vitro replicates (n=2), and all experiments were replicated at least twice. Graphs show mean±s.d. (FIG. 5B). * P<0.05, by one-way ANOVA with Tukey's multiple comparisons. Abbreviations: PI: phosphatidylinositol; PE-O: akylphosphatidylethanolamine; PE: phosphatidylethanolamine; HexCer: hexosylceramide; PG: phosphatidylglycerol; PS: phosphatidylserine; PC-O: alkylphosphatidylcholine; PS-O: alkylphosphatidylserine; LNAPE: lyso-N-acyl-phosphatidylethanolamine; LPC: lysophosphatidylcholine; Cer: ceramide; LPE: lysophosphatidylethanolamine.

FIG. 6 shows the results of in vitro Screening. Specifically, FIG. 6A shows representative images of the IL4: GFP expression obtained from 4get splenocyte cultures at day 5 using the Celigo plate imaging system. FIG. 6B shows representative images of the IL4: GFP expression obtained from 4get splenocyte cultures at day 5 using the Cellomics HTS. FIG. 6C shows a comparison of rSEA batches in a splenocyte culture system to expand CD45⁺ cells. FIG. 6D shows the reproducibility between batches of rSEA to stimulate IL4: GFP⁺CD4⁺ cells in vitro. Statistical tests represent all in vitro replicates, and all experiments were replicated at least twice, excluding (FIG. 6A). Graphs show mean±s.d. (FIGS. 6B-6D), n=3. * P<0.05, ** P<0.01, *** P<0.001, **** P<0.0001 by one-way ANOVA with Tukey's multiple comparisons (FIGS. 6B-6D).

FIG. 7 shows a comparison of SEA vs rSEA treated muscles. FIG. 7A shows gene expression of pro-inflammatory associated genes of 1-week post-VML muscles comparing SEA or rSEA, normalized to saline treated controls. FIG. 7B shows bulk RNASeq comparing SEA and rSEA. Statistical tests represent all biological replicates. Graphs show mean±s.d., n=3. * P<0.05, ** P<0.01, *** P<0.001, **** P<0.0001 by two-way ANOVA with Sidak's multiple comparisons.

FIG. 8 shows local versus systemic administration of rSEA. FIG. 8A shows a representative flow cytometry plots of IL4: GFP⁺CD4⁺ of local vs. systemic rSEA administration in muscle 1-week post-VML and respective percentages in 4get IL4: GFP reporter mice. FIG. 7B shows flow cytometry populations for myeloid populations in the muscle post-injury and treatment. FIG. 1C shows 1-week muscle injury ICS IL-4 cytokine staining in C57BL/6 mice. Statistical tests represent all biological replicates, except when otherwise noted. Graphs show mean±s.d. * P<0.05, ** P<0.01, *** P<0.001, **** P<0.0001 by one-way ANOVA with Tukey's multiple comparisons.

FIG. 9 shows Treg Populations in the iLNs after rSEA treatment of muscle injuries.

FIG. 9A shows FoxP3⁺ populations in 1-week post-VML in the draining inguinal lymph nodes. FIG. 9B shows flow cytometry intracellular staining of cytokines (ICS) in iLNs taken from C57BL/6 mice treated with rSEA, harvested 1-week after injury and treatment. Data are means±s.d., n=3-4. Statistical tests represent all biological replicates, except when otherwise noted. Graphs show mean±s.d. * P<0.05, ** P<0.01, *** P<0.001, **** P<0.0001 by one-way ANOVA with Tukey's multiple comparisons (a), and unpaired two-tailed Student's t-test (FIG. 9B).

FIG. 10 shows iLN and B Cell Responses to rSEA Treatment. FIG. 10A shows flow cytometry counts and % population of CD19⁺ cells in the iLNs at 1-week post-injury and rSEA treatment. FIG. 10B shows flow cytometry % population of B220⁺CD19⁺ cells in 4get mouse 1-week post-injury and treatment with rSEA. Statistical tests represent all biological replicates. Graphs show mean±s.d., n=3-4. * P<0.05, ** P<0.01, *** P<0.001, **** P<0.0001 by one-way ANOVA with Tukey's multiple comparisons (FIG. 10A), and unpaired two-tailed Student's t-test (FIG. 10B).

FIG. 11 shows in vitro B Cell Responses to rSEA Treatment. Specifically, flow cytometry total counts and % populations of CD19⁺ cells from in vitro splenocyte cultures taken from live CD45⁺ cells. Statistical tests represent all technical replicates. Graphs show mean±s.d., n=3. * P<0.05, ** P<0.01, *** P<0.001, **** P<0.0001 by one-way ANOVA with Tukey's multiple comparisons.

FIG. 12 shows macrophage populations in 1-week muscle post-rSEA treatment.

FIG. 12A shows representative flow cytometry plots of the CD206⁺ and CD86⁺ populations in muscle 1-week post-VML and rSEA treatment. FIG. 12B shows flow cytometry population cell counts of F4/80^{Hi+}MHCII⁺ cells as percentages of CD11b+ cells. FIG. 12C shows flow cytometry population cell counts of F4/80^{Hi+}SiglecF^{Neg}CD206⁺ or CD86⁺ cells and their percent of CD45⁺ live. FIG. 12D shows 1-week post-injury muscle gene expression of macrophage and eosinophil associated genes after rSEA treatment. FIG. 12E shows 1-week gene expression of injured muscle for skeletal muscle regeneration associated genes with rSEA treatment. Statistical tests represent all biological replicates. Graphs show mean±s.d., n=4. * P<0.05, ** P<0.01, *** P<0.001, **** P<0.0001 by unpaired two-tailed Student's t-test (FIGS. 12B and 12C), and two-way ANOVA with Sidak's multiple comparisons (FIG. 12D).

FIG. 13 shows gamma Delta T cell responses in muscle and iLN with rSEA treatment. FIG. 13A shows post-injury muscle γδ T cell total cells and percentages kinetics.

FIG. 13B shows iLN γδ T cell counts and percentages after muscle injury and treatment. FIG. 13C shows iLN IL17A⁺γδ counts and percentages 1-week post-injury of the muscle and treatment. Statistical tests represent all biological replicates. Graphs show mean±s.d., n=3-4. * P<0.05, ** P<0.01, *** P<0.001, **** P<0.0001 by two-way ANOVA with Sidak's multiple comparisons (FIGS. 13A and 13B), and unpaired two-tailed Student's t-test (FIG. 13C).

FIG. 14 shows T helper type 17 responses in the spleen with rSEA treatment. FIG. 14A shows IL17A⁺CD4⁺ total cells and % of CD4 in spleen 1-week post-injury and treatment. FIG. 14B shows IL4⁺CD4⁺ total cells and % of CD4 in spleen 1-week post-injury and treatment. Statistical tests represent all biological replicates. Graphs show mean±s.d., n=4. * P<0.05, ** P<0.01, *** P<0.001, **** P<0.0001 by unpaired two-tailed Student's t-test (FIGS. 14A and 14B).

FIG. 15 shows Vitrified gel development and delivery for rSEA. FIG. 15A shows a graphical synopsis of vitrified gel and packaging of rSEA. FIG. 15B shows representative images of SIS-vitrigel characterization in H&E (gross) and TEM (micro-architecture). FIG. 15C (left), shows swelling ratio comparison of various vitrified gels and hydrogel sources, the (right) assessment of storage modulus (G′) in Pascals, and values for vitrified gels (SIS, vit) and hydrogels (SIS, H). FIG. 15D shows the characterization of collagen content (% of dry mass) in vitrigels (left) and the sGAG content of the source material (particle form) compared to post-vitrification. FIG. 15E shows the biomechanical assessment of SIS-vitrigels in comparison to hydrogel forms for storage modulus, loss modulus, and viscosity in reference to % strain and angular frequency, respectively. FIG. 15F shows 1-week post-VML muscle gene expression of Il4 with the indicated treatment and normalized to saline treated control muscles (graph represents multiple independent experiments combined). Graphs show mean±s.d., n=3. * P<0.05, ** P<0.01, *** P<0.001, **** P<0.0001 by two-way ANOVA with Sidak's multiple comparisons (c, d-right). Scale bars: 200 μm (FIG. 15B, left), and 100 nm (FIG. 15B, right).

FIG. 16 shows injured Cornea immune populations with rSEA treatment. FIG. 16A shows representative flow cytometry plots of immune cell populations in injured cornea 1-week post-injury and % populations of CD45⁺ Live cells. Flow cytometry populations for T helper cell populations in the spleen 1-week post-injury and treatment. Flow Data generated by pooling 6 corneas to represent 1 sample (n=5). FIG. 16B shows the gene expression of cornea samples 1-week post-injury and treatment with rSEA or saline (n=4). Graphs show mean±s.d. * P<0.05, ** P<0.01, *** P<0.001, **** P<0.0001 by Student's t-test (FIG. 16A), and two-way ANOVA with Sidak's multiple comparisons (FIG. 16B).

FIG. 17 shows gating schemes for flow cytometry of various immune cell populations.

FIG. 18 shows rSEA treatment promotes a pro-regenerative type 2 immune microenvironment after muscle injury. Mice had a partial quadriceps resection to create volumetric muscle loss (VML) injury and received the indicated treatments administered locally at the time of resection. FIGS. 18A and 18B shows muscle tissue expression of selected T_{H}2 and T_{H}1 genes assessed by qRT-PCR 1-week post-VML and treatment with saline, unfractionated soluble SEA extract and fractionated SEA formulation (regenerative SEA, rSEA). FIG. 18C shows Il4 gene expression of iLNs from VML injured mice at different time points after rSEA treatment. FIG. 18D shows flow cytometric quantitation of post-injury muscle eosinophils, defined by co-expression of Siglec F and CD11b, over time with rSEA treatment. FIG. 18E shows muscle expression of selected type 2 genes after saline vs rSEA treatment of Wild Type (WT) and GATA1 KO mice. FIG. 18F shows a volcano Plot of differential expression from bulk-RNA sequencing of muscle 1-week post-injury and rSEA treatment referenced to saline treated injuries by EdgeR analysis. FIG. 18G shows flow cytometry of muscle CD3⁺CD4⁺ cells from IL4-reporter (4get) mice treated with either saline or rSEA. FIG. 18H shows muscle IFNγ⁺ production by various immune cell types, assayed by intracellular cytokine staining. FIG. 18I shows gene expression profile of sorted CD3⁺ T cells from muscle 1-week post-injury, comparing saline and rSEA. FIG. 18J shows CD4⁺Foxp3⁺ (Tregs) in the muscle 5 and 7 days after treatment with either saline or rSEA.

FIG. 18K shows flow plots of iLNs from 4get mice draining VML treated one week with saline or rSEA. FIG. 18L shows iLN gene expression 1-week after muscle injury and treatment. FIG. 18M shows iLN IFNγ production by various cell types assayed by ICS. FIG. 18N shows Gene expression profiling of CD11b⁺F4/80^{+Hi} macrophages sorted from muscle one week after saline vs rSEA treatment. Statistical tests represent all biological replicates, and all experiments excluding (FIG. 18F, FIG. 18I, 18M) were replicated at least twice. Graphs show mean±s.d. (FIGS. 18A-18D, 18G-18H, 18J-18M) and mean±SEM, n=3-5 (e). * P<0.05, ** P<0.01, *** P<0.001, **** P<0.0001 by unpaired two-tailed Student's t-test (FIG. 18G, 18K), and two-way ANOVA with Sidak's multiple comparisons. For volcano plot differential expression (FIG. 18F, FIG. 18I, FIG. 18N), EdgeR analysis was performed for bulk-sequencing FDR (FIG. 18F), and sorted cell NanoString analysis FDR values determined by the Benjamini-Yekutieli method (FIG. 18I, FIG. 18N). Dashed lines (FIG. 18F) represent −Log₁₀(FDR)<0.05 & 0.01, and in (FIG. 18I, 18N) represent −Log₁₀(Adj. P-value) significance determined by nCounter software (adj. P<0.5, 0.1, 0.05, & 0.01, respectively).

FIG. 19 shows rSEA promotes skeletal muscle repair and decreases fibrosis and type 3 immune responses. FIG. 19A shows 1-week post-injury expression of genes involved in muscle regeneration and associated with TH17 cells in mice treated with saline or rSEA. FIG. 19B shows 6-week post-injury muscle histology treated with either saline or rSEA, stained with Masson's trichrome, picrosirius red, and immunofluorescent staining [nuclei=DAPI (purple), mature muscle=dystrophin (green)]. Arrows indicate site of original muscle resection. FIG. 19C shows muscle gene expression levels of adipose browning associated genes at 1-week and 3-weeks post-injury and treatment with saline or rSEA. FIG. 19D shows treadmill exhaustion functional testing of mice at 6-weeks post-injury and treatment with saline or rSEA. FIG. 19E shows gene expression of various fibrosis-associated collagens at 6-weeks post-injury and treatment with saline or rSEA. FIG. 19F shows the percentage of CD4 cells expressing IL-17A at 1-, 3- and 6-week after injury and treatment with saline or rSEA as determined by ICS. FIG. 1919G shows the percentage of CD4 cells expressing IL-17A by ICS in iLN 1-week post-muscle-injury and treatment with saline or rSEA. FIG. 19H shows the percentage of γδ T cells expressing IL-17A in muscle at 1-week post-injury and treatment. FIG. 19I shows the percentage of γδ T cells expressing IL-17A in iLN at 1-week after injury and treatment with saline or rSEA. FIG. 19J shows a production summary and gross image of vitrified SIS-ECM combined with rSEA. FIG. 19K shows the gene expression of Il4 in muscle for 1-, 3-, and 6-weeks post-injury and treatment with either saline, rSEA, pure vitrigel or vitrigel formulated with rSEA. FIG. 19L shows transverse histological sections of muscles stained with Masson's trichrome 6-weeks post-injury and treatment with either saline, pure vitrigel and vitrigel formulated with rSEA. Statistical tests represent all biological replicates, and all experiments were replicated at least twice. Graphs show mean±s.d. (FIG. 19A, FIG. 19C-FIG. 19I, FIG. 19K), n=3-4. * P<0.05, ** P<0.01, *** P<0.001, **** P<0.0001 by two-way ANOVA with Sidak's multiple comparisons. Scale bars: 100 μm (FIG. 19B), 1 cm (FIG. 19J), and 1 mm (FIG. 19L).

FIG. 20 shows rSEA immunotherapy promotes repair in articular joint injuries. FIG. 20A shows ACLT injury model timeline. FIG. 20B shows representative images from Safranin-O stains of uninjured joints (no surgery) or injured joints treated with vehicle or rSEA 4 wks post-ACLT. Arrows indicate where cartilage damage has occurred. FIG. 20C shows OARSI scoring of uninjured joints or injured joints treated with vehicle or rSEA 4 wks post-ACLT. FIG. 20D shows articular joint gene expression of type 2, type 3 immune genes and cartilage extracellular matrix genes Aggrecan (Acan), Type 2 collagen (Col2a1), and Lubricin (Prg4) 4-weeks post-ACLT, treated with vehicle or rSEA. FIG. 20E shows iLN gene expression of type 2 and type 3 immune genes 4-weeks post-ACLT. FIG. 20F shows hot plate reaction times and weight bearing assessments in mice without injury or 4-weeks post-ACLT treated with vehicle (saline) or rSEA. Statistical tests represent all biological replicates, and all experiments were replicated at least twice. Graphs show mean±s.d. (FIG. 20C-20E), n=4-5, box and whisker plot with median as central line, and ‘+’ designates mean (FIG. 20C). * P<0.05, ** P<0.01, *** P<0.001, **** P<0.0001 by one-way ANOVA with Tukey's multiple comparisons (FIG. 20C, 20F), two-way ANOVA with Sidak's multiple comparisons (FIG. 20D, 20E). Scale bars: 100 μm (FIG. 20B).

FIG. 21 shows rSEA immunotherapy promotes repair in corneal injury. FIG. 21A shows cornea debridement injury model timeline. FIG. 21B shows corneal gross images and scar ratio assessment at 2-weeks post-injury treated with saline vehicle or rSEA, two study replications combined. FIG. 21C shows flow cytometry populations isolated from the cornea 1-week post-injury and treatment with saline or rSEA. FIG. 21D shows corneal expression of genes associated with scar vascularization at 1-week post-injury and treatment with saline or rSEA. FIG. 21E shows representative flow plots of 2-week post-injury 4get cervical LNs (cvLN) and % TH2 populations at 1-week vs. 2-weeks post-injury and treatment with saline or rSEA. FIG. 21F shows ICS from cvLN of TH1, TH17, and IFNγ⁺CD8⁺ T cells (%) at 1-week & 2-weeks post injury and treatment with saline or rSEA. FIG. 21G shows the representative gross images of wounded corneas from GATA1 KO mice treated 2-weeks with saline or rSEA and scar area ratio assessment. FIG. 21H shows immunofluorescent staining of nuclei (DAPI, blue) and α-SMA (green) on corneas 2-weeks post-injury and treatment with saline or rSEA in WT vs. GATA1 KO mice. FIG. 21I shows cvLN ICS for IL-4 in WT vs. GATA1 KO mice 2-weeks post-injury and treatment with saline or rSEA. Statistical tests represent all biological replicates, and all experiments were replicated at least twice. Graphs show mean±s.d. (FIG. 21B-21F, FIG. 21G, 21I), n=4-6. * P<0.05, ** P<0.01, *** P<0.001, **** P<0.0001 by One-way ANOVA with Tukey's multiple comparisons (FIG. 21B), and two-way ANOVA with Sidak's multiple comparisons (FIG. 21D-21F, 21G, 21I).

FIG. 22 shows a comparison of the process used to prepare the helminth regenerative compositions of the present disclosure containing a mixture of a soluble fraction (containing at least one helminth egg antigen) and an insoluble fraction derived from one or more helminth eggs compared to other processes known in the art for obtain helminth soluble egg antigen (SEA).

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other aspects “comprising,” “consisting of” and “consisting essentially of,” the aspects or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “helminth” refers to a parasitic worm or nematode. Any helminth that induces a type 2 immune response in a subject can be used in the present disclosure. In some aspects, the helminth can be from the Ascaris species (e.g., such as Ascaris lumbricoides), Nippostrongylus brasiliensis, Enterobuis vermicularis, Trichuris trichiura, Ancylostroma duodenale, Necator americanus, Strongyloides stercoralis, and Trichinella spiralis. In other aspects, the helminth is a platyhelminth from trematodes and cestodes, such as Fasciolopsis, Echinostoma and Heterophyes species, Clonorchis sinensis, Oplishorchis viverrini, Opisthorchis felineus, Fasciola hepatica, Schistosoma species (e.g., Schistosoma mansoni), Diphyllobothrium species, Taenia saginata, Taenia solium and Hymenolepsis nana. In yet other aspects, the helminth is from Trichuris muris, Trichinella spiralis, Nippostronglylus prasiliensis, Heligmonsomoides polygyrus, Hymenolepsis nanan, Angiostrongylus species, Trichuris suis, Ascaris suum, Trichuris vulpis, Toxocara species and Pseudoterranova species. In still other aspects, the helminth is a filarial parasite or lung fluke. In yet other aspects, the helminth is Schistosoma mansoni.

As used herein, the phrases “insoluble fraction derived from one or more helminth eggs” or “insoluble portion derived from one or more helminth eggs” as used interchangeably herein, refer to one or more lipids derived or obtained from one or more helminth eggs. In some aspects, the one or more lipids are purified and/or filtered from the one or more helminth eggs. Examples of such lipids include one or more prostaglandin D2 (PGD2), prostaglandin E2 (PGE2), 5-hydroxyeicosatetraenoic (HETE), 15-HETE, linoleic acid, arachidonic acid, docosahexaenoic acid, 5S,18R-dihydroxy-6E,8Z,11Z,14Z,16E-eicosapentaenoic acid, 10S,17S-dihydroxydocosa-4Z,7Z,11E,13Z,15E,19Z-hexaenoic acid, (4Z,7Z,10R,11E,13E,15Z,17S,19Z)-10,17-dihydroxydocosa-4,7,11,13,15,19-hexaenoic acid, lysophosphatidylcholine, or any combinations thereof.

In some aspects, the insoluble fraction derived from one or more helminth eggs comprises at least one of about 0.1% to about 20% PGD2, about 0.1% to about 20% PGE2, about 0.1% to about 20% of 5-hydroxyeicosatetraenoic acid (HETE), about 0.1% to about 20% of 15-HETE, about 0.1% to about 20% of at least one linoleic acid, about 0.1% to about 20% of arachidonic acid, about 0.1% to about 20% of docosahexaenoic acid, about 0.1% to about 20% of 5S,18R-dihydroxy-6E,8Z,11Z,14Z,16E-eicosapentaenoic acid, about 0.1% to about 20% of 10S,17S-dihydroxydocosa-4Z,7Z,11E,13Z,15E,19Z-hexaenoic acid about 0.1% to about 20% of (4Z,7Z,10R,11E,13E,15Z,17S,19Z)-10,17-dihydroxydocosa-4,7,11,13,15,19-hexaenoic acid, about 0.1% to about 20% of lysophosphatidylcholine, and any combinations thereof. In other aspects, the insoluble fraction derived from one or more helminth eggs and comprises about 0.1% to about 20% PGD2, about 0.1% to about 20% PGE2, about 0.1% to about 20% of 5-hydroxyeicosatetraenoic acid (HETE), about 0.1% to about 20% of 15-HETE, about 0.1% to about 20% of at least one linoleic acid, about 0.1% to about 20% of arachidonic acid, about 0.1% to about 20% of docosahexaenoic acid, about 0.1% to about 20% of 5S,18R-dihydroxy-6E,8Z,11Z,14Z,16E-eicosapentaenoic acid, about 0.1% to about 20% of 10S,17S-dihydroxydocosa-4Z,7Z,11E,13Z,15E,19Z-hexaenoic acid about 0.1% to about 20% of (4Z,7Z,10R,11E,13E,15Z,17S,19Z)-10,17-dihydroxydocosa-4,7,11,13,15,19-hexaenoic acid, and about 0.1% to about 20% of lysophosphatidylcholine.

“Promote,” “promotion,” and “promoting” refer to an increase in an activity, response, condition, disease, and/or other biological parameter. This can include, but is not limited to, the initiation of the activity, response, condition, and/or disease. This may also include, for example, a about a 5% increase in the activity, response, condition, and/or disease as compared to the native or control level. Thus, the increase can be about a 5, 10, 20, 30, 40, 50, 60, 70, 80, 90, 100%, or any amount of increase in between as compared to native or control levels.

“Regenerative” or “regeneration” as used herein refers to the renewal, re-growth, and/or restoration of a body or body part, such as, for example, a tissue, after injury or as a normal bodily process. In contrast to scarring, tissue regeneration involves the restoration of the body or body part to its original structural, functional, and physiological condition. In some aspects, the restoration can be partial or complete, meaning about 5%, about 10%, about 20%, about 25%, about 30%, about 40%, about 50%, about 60%, about 70%, about 75%, about 80%, about 90% or about 100% renewal, re-growth, or restoration, or any amount of restoration in between as compared to native and/or control levels. For example, in the case of a skin injury, tissue regeneration can involve the restoration of hair follicles, glandular structures, blood vessels, muscle, or fat. In the case of a brain injury, tissue regeneration can involve maintenance or restoration of neurons. In some aspects, tissue regeneration involves the recruitment and differentiation of stem cells to replace the damaged cells. As used herein, a “stem cell” refers to an undifferentiated cell found among differentiated cells in a tissue, or introduced from an external source for example, embryonic stem cells, adult bone marrow stem cells, that can renew itself and differentiate to yield the major specialized cell types of the tissue. The primary roles of stem cells in a living organism are to maintain and repair the tissue in which they are found. The phrase “stem cell differentiation” refers to the the process whereby an unspecialized cell (e.g., stem cell) acquires the features of a specialized cell such as a skin, neural, heart, liver, or muscle cell. In the case of a brain injury, tissue regeneration can involve the differentiation of stem cells into neurons.

As used herein, the phrases “soluble fraction derived from one or more helminth eggs” or “soluble portion derived from one or more helminth eggs” as used interchangeable herein refers to one or more egg antigens (e.g., proteins) derived or obtained from one or more helminth eggs. Examples of such antigens include one or more omega-1, IPSE/alpha-1, secretory glycoprotein kappa-5, smp40, Histone H2A, fructose-bisphosphate aldolase, lacto-N-fucopentaose-III (LNFPIII), lacto-n-Neotetraose (LNnT), or any combinations thereof.

In some aspects, the antigen is one or more of about 2% to about 50% of omega-1, about 2% to about 50% of about IPSE/alpha-1, about 2% to about 60% of secretory glycoprotein kappa-5, about 1% to about 40% of smp40, about 5% to about 70% of Histone H2A, about 10% to about 70% of fructose-bisphosphate aldolase, about 1% to about 50% of LNFPIII, about 1% to about 50% of LNnT, or any combinations thereof. In other yet aspects, the antigen is from about 2% to about 50% of omega-1, about 2% to about 50% of about IPSE/alpha-1, about 2% to about 60% of secretory glycoprotein kappa-5, about 1% to about 40% of smp40, about 5% to about 70% of Histone H2A, about 10% to about 70% of fructose-bisphosphate aldolase, about 1% to about 50% of LNFPIII, and about 1% to about 50% of LNnT.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some aspects, the subject may be a human or a non-human. In some aspects, the subject is a human. The subject or patient may be undergoing other forms of treatment. In some aspects, the subject is a human that may be undergoing other forms of treatment.

“Tissue” as used herein refers to groups of cells that have a similar structure and act together to perform a specific function. Examples of “tissue” include, muscle, an organ (e.g., brain, eye, heart, liver, pancreas, stomach, lung, large intestine, small, intestine, bladder, kidneys, reproductive organs, or any combinations thereof), cartilage, a ligament, skin, bone, nervous tissue, corneal tissue, a lens, or any combination thereof. In some aspects, the tissue is muscle. In other aspects, the tissue is an organ. In yet other aspects, the tissue is cartilage. In still yet other aspects, the tissue is a ligament. In yet other aspects, the tissue is skin. In still other aspects, the tissue is bone. In still other aspects, the tissue is nervous tissue. In still yet other aspects, the tissue is corneal tissue.

As used herein, “tissue injury” or “injury to a tissue” as used interchangeable herein, refers to an injury to a tissue that can result from, for example, a scrape, cut, laceration wound, crush wound, compression wound, stretch injury, bite wound, graze, bullet wound, explosion injury, body piercing, stab wound, burn wound, wind burn, sun burn, chemical burn, surgical wound, surgical intervention, medical intervention, host rejection following cell, tissue grafting, pharmaceutical effect, pharmaceutical side-effect, bed sore, radiation injury, cosmetic skin wound, internal tissue (e.g., organ) injury, disease process (e.g., asthma, cancer), infection, infectious agent, developmental process, maturational process (e.g., acne), genetic abnormality, developmental abnormality, environmental toxin, allergen, scalp injury, facial injury, jaw injury, foot injury, toe injury, finger injury, bone injury, sex organ injury, joint injury, excretory organ injury, eye injury, corneal injury, muscle injury, adipose tissue injury, lung injury, airway injury, hernia, anus injury, piles, ear injury, retinal injury, skin injury, abdominal injury, arm injury, leg injury, athletic injury, back injury, birth injury, premature birth injury, toxic bite, sting, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self mutilation, shock, traumatic soft tissue injury, spinal cord injury, spinal injury, sprain, strain, tendon injury, ligament injury, cartilage injury, thoracic injury, tooth injury, trauma, nervous system injury, aging, aneurism, stroke, digestive tract injury, infarct, ischemic injury, or any combination thereof.

By “treat” or “treatment” is meant a method of reducing the effects of a wound, injury, disease and/or condition. Treatment can also refer to a method of reducing the underlying cause of the wound, injury, disease and/or condition itself rather than just the symptoms. The treatment can be any reduction from native levels and can be but is not limited to the complete ablation of the disease, condition, or the symptoms of the disease or condition.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions of the Present Disclosure

In one aspect, the present disclosure relates a regenerative composition comprising a mixture of a soluble fraction and an insoluble fraction derived or obtained from one or more helminth eggs. The soluble fraction comprises at least one helminth egg antigen. The insoluble fraction comprises at least one lipid. In yet other aspects, one or both of the soluble fraction and the insoluble fraction are purified and/or filtered. In yet other aspects, the regenerative compositions comprise a mixture of a soluble fraction and an insoluble fraction derived or obtained from at least one egg derived from S. mansoni (such a composition is referred to herein as “rSEA”). It has been surprisingly discovered that the compositions of the present disclosure can be used to as pro-regenerative immunotherapies that can be used in promoting wound and/or tissue repair. It has also been found that when the regenerative compositions of the present disclosure are used in wound and/or tissue repair, the repaired wound and/or tissue exhibits reduced scar tissue and/or fibrotic tissue formation when compared to other compositions (such as extracellular matrix biomaterials) used for treating wounds and/or injuries to tissue. Moreover, the compositions of the present disclosure have been found increase IL-4 and decreases or reduces IL-17.

The helminth regenerative compositions comprising the mixture of a soluble fraction and an insoluble fraction is prepared using a unique process or method. Helminth eggs for use in the process can be obtained from any source known in the art. For example, the helminth eggs can be obtained from a commercially available source. Commercial sources of helminth eggs include, but are not limited to, Symmbio, Au Naturel, Biome Restoration, Ltd., etc. Alternatively, the eggs can be isolated from an animal infected with S. mansoni, such as a mouse, rat, rabbit, sheep, cow, cat, dog, cow, human, etc., using routine techniques known in the art.

The eggs are homogenized to produce a mixture using routine techniques known in the art (e.g., by using a homogenizer). In some aspects, the eggs are homogenized to greater than about 90%, greater than about 91%, greater than about 92%, greater than about 93%, greater than about 94%, greater than about 95%, greater than about 96%, greater than about 97%, greater than about 98%, or greater than about 99%. In some aspects, the eggs are 100% homogenized.

Once homogenized, the mixture is centrifuged using routine techniques known in the art. The centrifugation produces three different fractions or layers of the mixture: (a) a top lipid fraction or layer; (b) a middle soluble egg antigen fraction or layer; and (c) bottom egg shell fraction or layer. The bottom egg shell fraction or layer is then removed using routine techniques known in the art. The lipid fraction or layer and the soluble egg antigen fraction or layer is retained.

The lipid fraction or layer and the soluble egg antigen fraction or layer is then ultra-centrifuged using routine techniques known in the art. For example, the lipid fraction or layer and the soluble egg antigen fraction or layer can be ultra-centrifuged at 100,000×g for about 90 minutes at 4° C. The conditions under which the ultra-centrifugation occurs is not critical and can be modified or adjusted using routine techniques known in the art. The ultra-centrifugation produces an insoluble supernatant (e.g., fraction) that forms as a top layer followed by a soluble supernatant (e.g., fraction). The insoluble and soluble supernatants are harvested, separated or removed from each other using routine techniques known in the art, such as by use of a pipet. Each of the separated insoluble and soluble fractions are filtered using a sterile filter using routine techniques known in the art.

The filtered insoluble and soluble fractions are then combined in desired ratios to produce the helminth regenerative soluble egg antigen compositions of the present disclosure. In some aspects, the filtered insoluble and filtered soluble fractions are combined in a ratio sufficient to reduce a proinflammatory response in a subject. As used herein, the term “sufficient to reduce a proinflammatory response in a subject” refers to that when the composition of the present disclosure is administered to the subject, at least the subject's IL-17 and/or interfering gamma levels are reduced or decreased. In some aspects, the subject's IL-17 and/or interfering gamma levels are reduced or decreased by 1%, 5%, 10%, 15%, 20%, 25%, 30%, 40%, or 50%. In yet other aspects, the filtered soluble fraction is combined with the filtered insoluble fraction in a ratio of about 9:1 to about 6:1. In some aspects, the ratio is about 6:1, about 6:2, about 6:3, about 6:4, about 6:5, about 6:6, about 7:1, about 7:2, about 7:3, about 7:4, about 7:5, about 7:6, about 7:7, about 8:1, about 8:2, about 8:3, about 8:4, about 8:5, about 8:6, about 8:7, about 8:8, about 9.1.

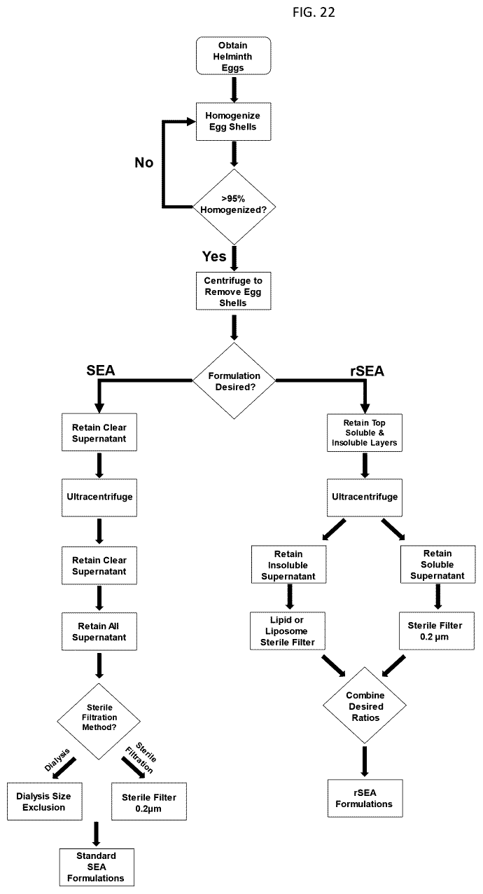

A comparison of the process used to prepare the regenerative compositions of the present disclosure (such as rSEA) with the process used in the art to produce compositions containing helminth soluble egg antigens (SEA) is shown in FIG. 22.

The regenerative compositions of the present disclosure have been found to stimulate or induce a type 2 immune response when administered to subjects suffering a wound and/or tissue injury and thus can be used to promote regeneration and repair of such wounds and/or tissue injuries. Thus, as discussed previously herein, these compositions can be used as pro-regenerative immunotherapies for wound and/or tissue repair in a subject in need of treatment thereof. Moreover, it has also been surprisingly discovered that when the compositions of the present disclosure are used for wound and/or tissue repair, the resulting repaired wound and/or tissue exhibits reduced scar tissue and/or fibrotic tissue formation when compared to other compositions used for treating wounds and/or injuries to tissue.

In addition to the mixture of a soluble fraction and an insoluble fraction derived from one or more helminth eggs, the compositions of the present disclosure can also contain at least one pharmaceutically acceptable carrier or excipient suitable for use in treating subjects (e.g., humans and/or animals) without undue adverse side effects (such as toxicity, irritation, and allergic response) commensurate with a reasonable benefit/risk ratio. The carrier or excipient would naturally be selected to minimize any degradation of the mixture of the soluble fraction and/or insoluble fraction and to minimize any adverse side effects in the subject, as would be well known to one of skill in the art. Examples of pharmaceutically acceptable carriers include those selected from the group consisting of water, polyhydric alcohols including alkylene glycols, (particularly propylene glycol) and glycerol; alcohols such as ethanol and isopropanol; polyalkylene glycols such as polyethylene glycol; other ointment bases such as petroleum jelly, lanolin, dimethylformamide, ethylene glycol, tetrahydrofurfuryl alcohol, cyclohexane, cyclohexanone, acetone, ethylether, N-dodecylazocyclo-heptan-2-one, methyldecylsulfoxide, dimethylacetamide and diethylfoluamide; and mixtures thereof.

It should be appreciated that the above list is not exclusive as the present disclosure also encompasses the use of pharmaceutically acceptable carriers other than those specifically mentioned. In some aspects, the composition may further contain thickeners, diluents, buffers, preservatives, surface active agents and the like in addition to the pharmaceutically acceptable carriers and the mixture of the soluble fraction and the insoluble fraction.

In further aspects, the composition can further comprise any known or newly discovered substance that can be administered to a wound, tissue injury, and/or site of inflammation that include antimicrobial agents, antiinflammatory agents, anesthetics, and the like. For example, the compositions of the present disclosure can further comprise one or more of classes of antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone), Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), chemotherapy (e.g. anti-cancer drugs such as but not limited to Altretamine, Asparaginase, Bleomycin, Busulfan, Carboplatin, Carrnustine, Chlorambucil, Cisplatin, Cladribine, Cyclophosphamide, Cytarabine, Dacarbazine, Diethylstilbesterol, Ethinyl estradiol, Etoposide, Floxuridine, Fludarabine, Fluorouracil, Flutamide, Goserelin, Hydroxyurea, Idarubicin, Ifosfamide, Leuprolide, Levamisole, Lomustine, Mechlorethamine, Medroxyprogesterone, Megestrol, Melphalan, Mercaptopurine, Methotrexate, Mitomycin, Mitotane, Mitoxantrone, Paclitaxel, pentastatin, Pipobroman, Plicamycin, Prednisone, Procarbazine, Streptozocin, Tamoxifen, Teniposide, Vinblastine, Vincristine), anti-inflammatory agents (e.g. Alclofenac; Alclometasone Dipropionate; Algestone Acetonide; alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Morniflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents (e.g. Ethanolamines (like diphenhydramine carbinoxamine), Ethylenediamine (like tripelennamine pyrilamine), Alkylamine (like chlorpheniramine, dexchlorpheniramine, brompheniramine, triprolidine), other anti-histamines like astemizole, theophyline, loratadine, fexofenadine, Bropheniramine, Clemastine, Acetaminophen, Pseudoephedrine, Triprolidine).

Additionally, the compositions of the present disclosure may further comprise at least one extracellular matrix. In some aspects, the composition is co-formulated with at least one extracellular matrix. Examples of some extracellular matrices that can be used include PuraPly and PuraPlyAM from Organogenesis, Inc. and Cellvo™ from Stembiosys, AlloDerm Regenerative Matrix, Cymetra, Integra Dermal Regeneration Template, MiMidex materials (e.g., EPIFIX, EPICORD). These materials can also be combined with synthetic materials known in the art as well.

The compositions of the present disclosure can be in any form suitable for administration to a subject. Specifically, the compositions of the present disclosure can be contained or incorporated into one or more materials. For example, such materials can include materials used to treat wounds in which such materials are coated and/or impregnated with the compositions described herein. Examples of such materials which can be used to treat wounds include bandages, steri-strips, sutures, staples, grants (e.g., skin grafts) or any combinations thereof. For example, the material (e.g., bandage, steri-strip, suture, staple, graft, or combinations thereof) can be soaked, coated and/or dipped with or into the compositions described herein. Alternatively, the material can be coated with the composition described herein. Alternatively, the composition can be incorporated into the materials. For example, the composition can be incorporated into a hydrogel, such as a cross-linkable hydrogel system, such as the poly(lactic-co-glycolic acid) (PLGA) or polyurethane. In another aspect, the compsition can be incorporated into a vitrified gel (e.g., vitrigel). The hydrogel, vitrified gel, or hydrogel system can be fashioned into materials for treating wounds (e.g., bandage, steri-strip, suture, staple, graft, or combinations thereof). In yet another aspect, medical implants can be coated with the regenerative composition of the present disclosure. Such implants can be implanted into a subject for purposes of treatment. Examples of medical implants include, limb prostheses, breast implants, penile implants, testicular implants, artificial eyes, facial implants, artificial joints, heart valve prostheses, vascular prostheses, dental prostheses, facial prosthesis, tilted disc valve, caged ball valve, ear prosthesis, nose prosthesis, pacemakers, cochlear implants, and skin substitutes (e.g., porcine heterograft/pigskin, BIOBRANE, cultured keratinocytes).

The compositions of the present disclosure may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal, intramuscular injection or injection directly into the affected area requiring treatment, such as, but not limited to, muscle wound or arthritic joint. Injectables can be prepared in conventional forms, either as liquid solutions or suspensions, solid forms suitable for solution of suspension in liquid prior to injection, or as emulsions.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

In some aspects, when the compositions are applied topically, for a local effect, the delivery of the composition is made to the skin but can also include the delivery of the compositions into the nose and nasal passages through one or both of the nares. Suitable forms include ointments, lotions, creams, gels (e.g., poloxamer gel), drops, suppositories, sprays, liquids, powders, sprayable liquids, liquids that may be applied using a roll-on device, lacquers, and sustained release matrices of transdermal delivery devices such as patches. Administration of the composition by inhalant can be through the nose or mouth via delivery by a spraying or droplet mechanism. Delivery can also be directly to any area of the respiratory system (e.g., lungs) via intubation. Conventional pharmaceutical carriers, aqueous, powder or oily bases, thickeners and the like can used if desirable. Moreover, the compositions of the present disclosure can be administered, for example, in a microfiber, polymer (e.g., collagen), nanosphere, aerosol, lotion, cream, fabric, plastic, tissue engineered scaffold, matrix material, tablet, implanted container, powder, oil, resin, wound dressing, bead, microbead, slow-release bead, capsule, injectables, intravenous drips, pump device, silicone implants, or any bio-engineered materials.

In some aspects, the compositions of the present disclosure are formulated in such a way as to ensure that an effective amount of the mixture of the soluble fraction and insoluble fraction derived or obtained from one or more helminth eggs is present in the composition to elicit Type-2 immune response after administration or application thereof. More specifically, effective dosages and schedules for administering or application the compositions can be determined empirically, and making such determinations is within the skill in the art. The dosage ranges for the administration or application of the compositions are those sufficient enough to produce the desired effect in which the wound healing is achieved or effected. The dosage should not be so large as to cause adverse side effects, such as unwanted cross-reactions, anaphylactic reactions, and the like. Generally, the dosage will vary age, sex, weight and general condition of the subject, the severity of the wound being treated, the type of wound, the patient's immune state, route of administration or application, or whether other drugs are included in the regimen, and can be determined using routine techniques known in the art. The dosage can be adjusted by a clinician in the event of any counterindications. Dosages can vary, and can be administered or applied in one or more dose administrations daily, for one or several days. Guidance can be found in the literature for appropriate dosages for given classes of pharmaceutical products. The range of dosage largely depends on the application of the compositions herein, severity of condition, and its route of administration or application.

Following administration or application of the compositions of the present disclosure for promoting wound healing and/or tissue repair, the efficacy of the composition can be assessed in various ways well known to the skilled practitioner. For example, a person skilled in the art will understand that a composition disclosed herein is efficacious in promoting wound healing in a subject by observing that the composition can reduce scar tissue formation, reduce fibrotic tissue formation, improve tissue regeneration, and/or reduce inflammation in the subject following tissue injury using routine techniques known in the art.

The materials described above as well as other materials can be packaged together in any suitable combination as a kit useful for performing and/or aiding in the performance of the methods described below in Section 3. It beneficial for the kit components to be designed and adapted for use together in the below described methods. For example, if the kits are for promoting wound and/or tissue healing, the kit can contains one or more of the compositions of the present disclosure and in a pharmaceutically acceptable carrier. Such kits can also include gels, bandages, Millipore tapes, Medicated Q-tips, Sprays, props, Syrups, Liquids, Disposable tubes or pouches. The kits also can contain instructions for proper use and safety information of the product or formulation. The kits may contain dosage information based on the route of administration or application and method of administration or application as determined by a clinician.

### 3. Methods of the Present Disclosure

In yet another aspect, the present disclosure relates to a method of promoting wound healing in a subject that has suffered an injury. In this aspect, the method comprises administering to the subject in need of such treatment one or more of the herein provided compositions in a pharmaceutically acceptable carrier. In yet another aspect, the present disclosure also provides method of treating a subject with tissue injury. In this aspect, the method comprises administering to the subject (e.g., having an injury and/or damage to a tissue) one or more of the herein provided compositions in a pharmaceutically acceptable carrier.

The methods provided herein can reduce scar tissue formation in a subject following wound and/or tissue injury. By “scar tissue” is meant the fibrous (fibrotic) connective tissue that forms at the site of injury or disease in any tissue of the body, caused by the overproduction of disorganized collagen and other connective tissue proteins, which acts to patch the break in the tissue. Scar tissue may replace injured skin and underlying muscle, damaged heart muscle, or diseased areas of internal organs such as the liver. Dense and thick, it is usually paler than the surrounding tissue because it is poorly supplied with blood, and although it structurally replaces destroyed tissue, it cannot perform the functions of the missing tissue. It is composed of collagenous fibers, which will often restrict normal elasticity in the tissue involved. Scar tissue may therefore limit the range of muscle movement or prevent proper circulation of fluids when affecting the lymphatic or circulatory system. Glial scar tissue following injury to the brain or spinal cord is one of the main obstacles to restoration of neural function following damage to the central nervous system. A reduction in scar tissue can be assessed by the population of cell types within the injured site. For example, a reduction in glial scar tissue can be estimated by an increased ratio of neuronal to astrocytic cells. A reduction in scar tissue formation can be measured by a simple measurement of scar width or area of scar tissue. In addition, histological assessments can be made about the restoration of structural complexity within healed tissue in comparison to normal tissue.

The methods provided herein can restore normal tissue mechanical properties such as tensile strength following tissue injury in a subject. “Tensile strength” refers to the amount of stress or strain required to break the tissue or wound. The tensile strength of treated wounds can be about 60, 65, 70, 75, 80, 85, 90, 95, 100% that of uninjured tissue within about 3 months after treatment. Thus, also provided is a method of restoring tissue mechanical properties, including increasing tensile strength of a healed injury to approach or reach that of normal uninjured tissue, in a subject comprising administering to the subject one or more of the compositions of the present disclosure in a pharmaceutically acceptable carrier.

The methods provided herein can improve tissue regeneration following tissue injury in a subject. The methods can enhance stem cell differentiation following tissue injury in a subject. Enhanced stem cell differentiation can be measured by providing a clinically acceptable genetic or other means of marking endogenous or engrafted stem cells and determining the frequency of differentiation and incorporation of marked stem cells into normal tissue structures.

Additionally, the methods provided herein can reduce inflammation in a subject. A reduction in inflammation can be measured by a reduction in the density of inflammatory cell types such as, for example, monocytes or astrocytes. A reduction in inflammation can be measured by a reduction in the density of inflammatory cell types such as, for example, neutrophils, mast cells, basophils, and monocytes. A reduction in inflammation can be calculated by an in vivo measurement of neutrophil activity. In addition, factors like frequency of mast cell degranulation or measurement of histamine levels or levels of reactive oxygen species can be used as measurements of reduction in inflammation. The level of inflammation can also be indirectly measured by checking for transcription levels of certain genes by qRT-PCR for example, for genes like, Interferon-alpha, -beta and -gamma, Tumor Necrosis Factor-alpha, Interleukine 1 beta, -2, -4, -5, -6, -8, -12, -18, -23, -27, CD4, CD28, CD80, CD86, MHCII, and iNOS. Measurement of pro-inflammatory cytokine levels in the tissues and or bodily fluids of the subject including plasma can measure a reduction in inflammation.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and aspects disclosed herein. Having now described the present disclosure in detail, the same will be more clearly understood by reference to the following examples, which are merely intended only to illustrate some aspects and aspects of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

The material and methods for use in Example 2 is provided below.

### Materials and Methods

### Mice

Mice were housed and maintained in the Johns Hopkins Cancer Research Building animal facility in compliance with ethical guidelines outlined by the Animal Care and Use Committee (ACUC). All procedures performed on animals were approved by Johns Hopkins ACUC. Investigators involved with the studies were blinded whenever possible. All mice used in these studies shown below in Table 1 were maintained as Helicobacter negative.

TABLE 1

| Mouse Strain | Alternative Name | Background | Company | Stock# |
|---|---|---|---|---|
| C57BL/6J | — | — | The Jackson Laboratory | 000664 |
| BALB/CJ | — | — | The Jackson Laboratory | 000651 |
| 4Get | C.129-B4 [symbol] /J | BALB/cJ | The Jackson Laboratory | 004190 |
| ΔdblGATA | C.12951(B6)-Gata1 [symbol] /J | BALB/cJ | The Jackson Laboratory | 005653 |

[symbol]

indicates data missing or illegible when filed

### Volumetric Muscle Loss (VML) Model

The VML injury was performed in female mice as a bilateral surgical removal of the quadriceps femoris as previously described. A unilateral longitudinal incision measuring approximately 1.5 cm in length was made in the epidermis, dermis, and the underlying fascia above the muscle. Using sterilized microdissection scissors, a 3 mm×4 mm×4 mm full thickness segment of skeletal muscle was resected from each hindlimb. The remaining defect space was filled with 50 μL to 75 μL of vehicle (1×DPBS or treatment). Immediately after treatment the epidermis and dermis were closed using a wound clipper with 7 mm sterile wound clips (Roboz, USA).

### Anterior Cruciate Ligament Transection (ACLT) Model and Joint Evaluation

Post-traumatic osteoarthritis (PTOA) was induced in male mice by utilizing an anterior cruciate ACLT injury model in 10-week-old male C57BL/6j mice. SEA, rSEA, and various components were administered to the joint space of the operated knee via a 30-gauge needle or intraperitoneally. The joint cavity was opened in the sham group, but the ACL was not transected. Weight-bearing in mice was measured in the un-operated control animals and compared to ACLT animals receiving PBS control or rSEA therapy using an incapacitance tester (Columbus Instruments). The percentage if weight distributed on the ACLT limb was used as an index of joint discomfort in OA (55). The mice were positioned to stand on their hind paws in an angled box placed above the incapacitance tester so that each hind paw rested on a separate force plate. The force (g) exerted by each limb was measured. Three consecutive 3-sec readings were taken and averaged to obtain the mean score (64). To determine pain response times in post-injury and treated animals, mice were placed on an enclosed hotplate set to 55° C. The latency period for hind limb response (marked as jumping or paw-licking) was recorded as the response time before surgery and 4 weeks after surgery in all animal groups (55). At least three readings were taken per mouse and averaged to obtain the mean response time for each time point. After 4 weeks, animals were sacrificed, and mouse knees were fixed in 10% neutral-buffered formalin, decalcified for approximately 2 weeks in 10% EDTA at 4° C., step-wise dehydrated in EtOH, cleared in xylenes, and embedded in paraffin. 7 μm sections were taken throughout the joint, dried, and stained for proteoglycans with Safranin-O and Fast Green (Applied biosciences) per manufacturer's instructions. Evaluation of the cartilage damage was performed according to the Osteoarthritis Research Society International (OARSI) scoring system and was performed by blinded histological assessment the medial plateau of the tibia (65). Osteophytes on the tibial plateau were scored from 0 to 3, with 0 indicating no osteophytes or an osteophyte up to 100 μm in diameter; a score of 1 indicating an osteophyte measuring 100 μm to 200 μm in diameter; a score of 2 indicating an osteophyte of 200 μm to 300 μm in diameter; and a score of 3 indicating an osteophyte measuring more than 300 μm in diameter (64, 65).

### Corneal Debridement Surgery and Scar Quantification

All surgical procedures were performed under the guideline of the Johns Hopkins University Animal Care and Use Committee (ACUC). Male adult (8-12 weeks old) BALB/c mice, GATA1 KO mice, and IL4-IRES-eGFP (4get) mice were purchased from Jackson Labs. The corneal debridement wound was adapted with minor modifications from Stepp et al., 2014 (56). Mice were weighed and anesthetized with 90 mg/kg ketamine HCl (VetOne) with 10 mg/kg Xylazine HCl (VetOne) by injection. Proparacaine hydrochloride ophthalmic eye drops (Sandoz) were applied after the mice were sedated. The center area of the cornea was marked by a 1.5 mm biopsy punch, and the epithelium layer was removed within the area by a 1.5 mm flat blade (Fine Science Tools). After epithelium removal, a volume of 50 μL PBS solution with or without rSEA, were injected to the subconjunctival space of the wounded eye. After injection, drops of sterile PBS solution were applied to both wounded and unwounded eyes to keep the eyes moist until the mice were recovered from anesthesia. At 14 days post-surgery, the mice were euthanized, and the eye globes were collected. The picture of each globe was taken under surgical microscope (Nikon), and the scar areas and cornea areas were determined with ImageJ. The ratio was quantified as: scar ratio=Aₛ (scar area)/A_{c} (whole corneal area).

S. mansoni Egg Collection and Isolation of SEA

S. mansoni (from infected NMRI mice) reagents were provided by the NIAID Schistosomiasis Resource Center of the Biomedical Research Institute (Rockville, MD) through NIH-NIAID Contract HHSN272201700014I, supplied frozen at −80° C. Standard SEA was prepared according to standard operating procedures utilized by the center and based on Boros et. al (28). After thawing on ice in the dark, eggs were re-suspended in 4° C. 1×DPBS at a concentration of 100,000 eggs/mL and were homogenized on ice using a motorized pestle, or with a 2 mL dounce homogenizer (Kimble, USA). 95% to 100% of the eggs were disrupted, verified by visualization with a phase contrast microscope. The crude mixture was then centrifuged at 4° C. at 200×g for 45 minutes. The supernatant was retrieved and ultracentrifuged for 90 min at 100,000×g at 4° C. The entirety of the final supernatant was passed through a 0.22 μm sterile filter and stored at −80° C. Concentrations were determined using standard Bradford assays and the Qubit™ Protein Assay Kit (Invitrogen).

Isolation of rSEA Formulations

S. mansoni eggs were homogenized to isolate SEA as stated above with several modifications in the extraction process to generate rSEA. Initially the eggs are homogenized to 95% to 100%, verified by phase contrast microscopy. Centrifuged at 21,000×g for 45 minutes, then ultra-centrifuged at 100,000×g at 4° C. for 90 min. After ultra-centrifugation, an insoluble mixture that forms at the top layer is harvested and stored in sterile low-protein binding 1.5 mL Eppendorf tubes. The top half of the resulting soluble antigen volume is carefully removed by pipet, and sterile filtered using a low-protein binding 0.2 μm filter into a low-protein binding 1.5 mL tube. 900 μL of the soluble fraction is then combined with 100 μL of the lipid fraction that was sterile filtered using lipid 1.2 μm medical Supor disc filters (B Braun Medical, USA). The final mixture is then stored at −80° C. Concentrations were determined using standard Coomassie Bradford assays (ThermoFisher) and the Qubit Protein Assay Kit (Invitrogen).

### Lipid Analysis

Protein concentration of SEA and rSEA samples were measured prior to lipid analysis using a Qubit Protein Assay Kit (Invitrogen, Q33211). SEA or rSEA solutions at 2 mg/mL protein concentration were mixed 1:1 with chloroform, vortexed, and centrifuged at 15,000×g for 1 minute at 4° C. The organic phase of the resulting mixture was removed, dried with nitrogen gas, and resuspended in a 2:1:1 mixture of isopropyl alcohol/acetonitrile/water for LC-MS. Reversed-phase HPLC was performed with a C18 column (Phenomenex, 00D-4726-AN), MS was performed with a Bruker timsTOF Pro instrument, and post-run analysis was performed with Bruker MetaboScape software.

Preparation of Decellularized Extracellular Matrix from Porcine Small Intestines

Decellularized extracellular matrix (ECM) was produced from porcine small intestinal sub-mucosa (SIS) following procedures developed under Stephen Badylak and described in Keane, et al., with minor modifications (66). Fresh porcine small intestines were obtained from Wagner Meats (Maryland, USA), harvested from a five-year old animal. The tissues were thoroughly washed to remove debris and mechanically processed to remove mucosal, serosal, and muscular layers by scraping with sterile pyrogen-free plastics. The resulting tissue identified as SIS include the submucosa and basilar layers of the tunica mucosa, was treated using 0.1% peracetic acid (Sigma Aldrich) and 4% EtOH prepared type-1 sterile water in pyrogen-free plastics for 2 hrs while stirring. The ECM was then returned to neutral pH using serial washes of quality-1 water and sterile culture grade 1×DPBS. Upon return to neutral pH the samples were lash frozen in liquid nitrogen and lyophilized. All tissues were then cryo-milled in liquid nitrogen to particle mesh sizes approximately <400 μm and stored at −20° C. until use.

### Preparation of Vitrified Extracellular Matrix Hydrogels (Vitrigels)

To enable delivery of rSEA, a vitrified ECM was utilized to combine the benefits of ECM biomaterials with rSEA for enhancing pro-regenerative outcomes. SIS-ECM vitrigels were verified to enable a measurable release of biological payloads while acting as an immunomodulator for immune type 2 responses. SIS-ECM is digested with 1 mg of pepsin (Sigma Aldrich) and 10 mg of ECM with 0.01 HCl in type-1 water, covered, and stirred for 48 hrs at room temperature. The working solution is then cooled on ice and neutralized with 1 mL of a 60 mM HEPES and 0.05 mM NaOH solution, and therapeutics like rSEA are added immediately after the neutralization is confirmed by a pH of 7. Gelation is then allowed to occur at 37° C. for 2 hrs and then placed into a 40° C. vitrification chamber for 7 days. Just before implantation, the vitrified gels are hydrated with 100 μL to 200 μL and trimmed into 2 mm×2 mm pieces.

Gene Expression Tissue Processing and qRT-PCR

Harvested tissues were immediately placed into RNALater for at least 24 hrs at 4° C., transferred into TRIzol reagent (Thermo Fisher Scientific), flash frozen, and stored in a −80° C. freezer. For mRNA isolation, samples were homogenized using a Bead Ruptor 12 (OMNI International) using the highest speed setting for 3 rounds of 15 secs with 2.8 mm ceramic beads (OMNI International). RNA was isolated from whole tissue using TRIzol reagent and chloroform extraction. RNA was purified using Qiagen's RNeasy PLUS kits (mini-kit and micro-kit), with gDNA eliminator columns. All qRT-PCR was performed using TaqMan Gene Expression Master Mix (Applied Biosystems) and TaqMan probes according to manufacturer's instructions. Briefly, 2.5 μg of mRNA was used to synthesize cDNA using Superscript IV VILO Master Mix (ThermoFisher Scientific) utilizing manufacturer guidelines with a C1000 Touch Thermocycler (Bio-Rad). The cDNA concentration was set to 100 ng/well (in a total volume of 20 μL qRT-PCR reaction). The qRT-PCR reactions were performed on the StepOne Plus Real-Time PCR System and software (Applied Biosystems, ThermoFisher Scientific), as TaqMan single-plex FAM-MGB assays, TaqMan Gene Expression Master Mix, using manufacturer recommended settings for quantitative and relative expression. All qRT-PCR reactions were performed in 96-well MicroAmp Fast Optical Plates (Life Sciences). For tissue samples, B2m, Rer1, Hprt, and Ppia were used as endogenous controls (reference housekeeping genes), with samples normalized to the most stable endogenous control. Samples were normalized to vehicle treated (saline) controls, unless otherwise stated. All qRT-PCR data was analyzed using the Livak Method, wherein ΔΔCt values are calculated and reported as relative quantification values (RQ), established by the result of the 2^{-ΔΔCt} calculation (67). These results were further verified by analysis using the appliedbiosystems relative quantification online software (Thermo Fisher Scientific, ver. 2020.2.1-Q2-20-build4). RQ, same as fold-change (FC), values are represented by the geometric means with error bars representing the geometric standard deviation or by Log₂ (FC) wherein the data are displayed linearly as means with the error bars representing standard deviation. All qRT-PCR assays were completed within the laboratory at Johns Hopkins. Table 2 shows the murine TaqMan gene expression assay probes used.

TABLE 2

| Probe: | Assay ID: | Probe: | Assay ID: | Probe: | Assay ID: |
|---|---|---|---|---|---|
| B2m | Mm00437762_m1 | Gata3 | Mm00484683_m1 | Lyve1 | Mm00475058_m1 |
| Acan | Mm00545794_m1 | Hprt | Mm03024075_m1 | Myf5 | Mm00435125_m1 |
| Acta2 | Mm00725412_s1 | Igf1 | Mm00439560_m1 | Mymk | Mm00481256_m1 |
| Areg | Mm00437583_m1 | Ifng | Mm01168134_m1 | Myod1 | Mm00440387_m1 |
| Cd31 | Mm01242584_m1 | Il1b | Mm00434228_m1 | Myog | Mm00446194_m1 |
| Cd36 | Mm00432403_m1 | Il4 | Mm00445259_m1 | Pax7 | Mm01354484_m1 |
| Col1a1 | Mm00801666_g1 | Il5 | Mm00439646_m1 | Ppia | Mm02342430_g1 |
| Col2a1 | Mm01309565_m1 | Il6 | Mm00446190_m1 | Prg4 | Mm01284582_m1 |
| Col3a1 | Mm00802300_m1 | Il13 | Mm00434204_m1 | Ptgs2 | Mm00478374_m1 |
| Col4a2 | Mm00802386_m1 | Il17a | Mm00439618_m1 | Rer1 | Mm00471276_m1 |
| Col5a1 | Mm01332580_m1 | Il17f | Mm00521423_m1 | Tgfb2 | Mm00436955_m1 |
| Col6a5 | Mm01231908_m1 | Il23a | Mm00518984_m1 | Vegfc | Mm00437310_m1 |

### Tissue Preparation and Flow Cytometry

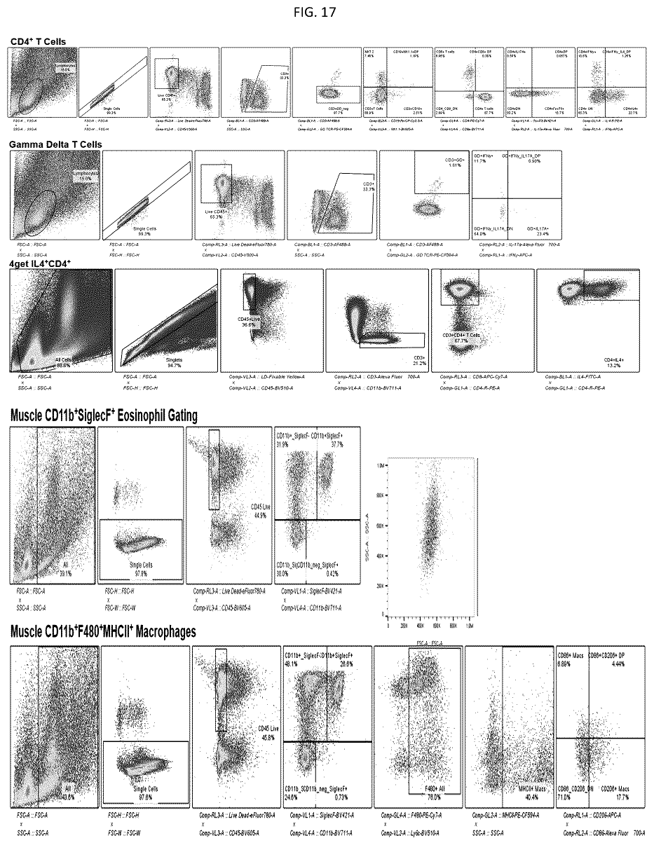

Tissue samples were obtained by cutting the quadriceps femoris muscle from the hip to the knee. Tissues were finely diced and digested for 45 min at 37° C. with 1.67 Wünsch U/mL (5 mg/mL) of Liberase TL (Roche Diagnostics, Sigma Aldrich) and 0.2 mg/mL DNase I (Roche Diagnostics, Sigma Aldrich) in RPMI-1640 medium supplemented with L-Glutamine and 15 mM HEPES (Gibco). The digested tissues were ground through 70 μm cell strainers (ThermoFisher Scientific) with excess RPMI-1640 (supplemented as before), and then washed twice with 1×DPBS. A discontinuous Percoll (GE Healthcare) density gradient centrifugation was used to enrich the leukocyte fraction (80%, 40%, and 20% layers) and to remove blood and debris from the muscle samples, centrifuged at 2,100×g for 30 min with the lowest acceleration, no brake, at room temperature. For intracellular staining, cells were stimulated for 4 hrs with Cell Stimulation Cocktail Plus Protein Transport Inhibitors (eBioscience) diluted in complete culture media (RPMI-1640 supplemented with 10% FBS, 15 mM HEPES, and 5 mM Sodium pyruvate). Cells were washed and surface stained, followed by fixation/permeabilization (Cytofix/Cytoperm, BD), and then stained for intracellular markers. Flow cytometry was performed using Attune N×T Flow Cytometer (ThermoFisher Scientific). Gating schemes are provided in FIG. 17. The enriched cells were washed and stained with the antibody panels as shown below in Table 3.

TABLE 3

| Mouse Pan-Immune Surface | | | | | | Monse Intracellular Cytokine Staining | | | | | |
|---|---|---|---|---|---|---|---|---|---|---|---|
| Filter | Marker | Fluorophore | Dilution | Clone | Source | Filter | Marker | Fluorophore | Dilution | Clone | Source |
| VL1 | Ly6G | PacBlue | 1-250 | 1AB | Biolegend | VL1 | Foxp3 | BV421 | 1-500 | MF-14 | Biolegend |
| VL2 | Ly6C | BV510 | 1-250 | HK1.4 | Biolegend | VL2 | CD45 | V500 | 1-150 | 30-F11 | BD |
| VL3 | CD45 | BV605 | 1-150 | 30-F11 | Biolegend | VL3 | NK1.1 | BV605 | 1-300 | PK136 | Biolegend |
| VL4 | CD8a | BV711 | 1-300 | 53-6.7 | Biolegend | VL4 | CD8a | BV711 | 1-300 | 53-6.7 | Biolegend |
| BL1 | CD4 | FITC | 1-250 | GK1.5 | Biolegend | BL1 | CD3 | AF488 | 1-200 | GK1.3 | Biolegend |
| BL3 | CD19 | PerCP-Cy5.5 | 1-250 | 6D5 | Biolegend | BL3 | CD19 | PerCpCy5.5 | 1-200 | 6D5 | Biolegend |
| GL1 | CD3 | PE | 1-300 | 17A2 | Biolegend | GL1 | IL-4 | PE | 1-200 | 11B11 | Biolegend |
| GL2 | SiglecF | PE-CF594 | 1-350 | E50-2440 | BD | GL2 | γδ | PE-594 | 1-300 | GL3 | Biolegend |
| GL4 | F4/80 | PE-Cy7 | 1-400 | BM8 | Biolegend | GL4 | CD4 | PE-Cy7 | 1-300 | GK1.5 | Biolegend |
| RL1 | CD11c | APC | 1-250 | N418 | Biolegend | RL1 | IFNγ | APC | 1-200 | XMG1.2 | Biolegend |
| RL2 | CD11b | AP700 | 1-400 | M1/70 | Biolegend | RL2 | IL-17a | AP700 | 1-200 | TC11-18H10.1 | Biolegend |
| RL3 | Viability | eFlour780 | 1-1000 | NA | ThermoFisher | RL3 | Viability | eFlour780 | 1-1000 | N/A | Thermo |

| Mouse 4get IL4: GFP Pan-Inmune | | | | | | Mouse Myeloid Panel | | | | | |
|---|---|---|---|---|---|---|---|---|---|---|---|
| Filter | Marker | Fluorophore | Dilution | Clone | Source | Filter | Marker | Fluorophore | Dilution | Clone | Source |
| VL1 | SiglecF | BV421 | 1-200 | E50-2440 | Biolegend | VL1 | CD86 | BV421 | 1-300 | GL1 | Biolegend |
| VL2 | Ly6c | BV510 | 1-150 | HK1.4 | Biolegend | VL2 | Ly6c | BV510 | 1-300 | HK1.4 | Biolegend |
| VL3 | CD45 | BV605 | 1-150 | 30-F11 | Biolegend | VL3 | CD45 | BV605 | 1-150 | 30-F11 | Biolegend |
| VL4 | CD8 | BV711 | 1-200 | 53-6.7 | Biolegend | VL4 | CD11b | BV711 | 1-400 | M1/70 | Biolegend |
| BL1 | IL-4 | GFP | N/A | N/A | Biolegend | BL1 | CD9 | FITC | 1-200 | MZ3 | Biolegend |
| BL3 | CD19 | BB700 | 1-250 | 1D3 | Biolegend | BL3 | — | — | — | — | — |
| GL1 | CD4 | PE | 1-300 | 11B11 | Biolegend | GL1 | CD301b | PE | 1-250 | URA-1 | Biolegend |
| GL2 | MCHII | PE-594 | 1-250 | M5/114.15.2 | Biolegend | GL2 | MHCII | PB-594 | 1-250 | M5/114.15.2 | BD |
| GL4 | F4/80 | PE-Cy7 | 1-200 | EMR1 | Biolegend | GL4 | F4/80 | PB-Cy7 | 1-250 | EMR1 | Biolegend |
| RL1 | CD206 | APC | 1-250 | C068C2 | Biolegend | RL1 | CD206 | APC | 1-250 | C068C2 | Biolegend |
| RL2 | CD11b | AF700 | 1-400 | M1/70 | Biolegend | RL2 | CD11c | AF700 | 1-200 | M1/70 | Biolegend |
| RL3 | Viability | Fixable Yellow | 1000 | N/A | Thermo | RL3 | Viability | eFlour780 | 1-1000 | NA | ThermoFisher |

### Fluorescence Activated Cell Sorting (FACS)

T cells (Live CD45⁺CD11b CD3⁺Singlets) and macrophages (Live CD45⁺CD11b⁺CD3-F4/80^{Hi} Singlets) were sorted from quadriceps femoris muscles 1-week post-injury. Tissue processing is the same as described above for flow cytometry, but without Percoll isolation. Only viability and surface staining were performed for FACS, and these experiments were performed using a BD FACSAria Fusion SORP. The cell sort gating scheme is provided in FIG. 17. Antibody clones and dilutions utilized for the sort are listed in Table 4 below.

TABLE 4

| Marker | Fluorophore | Dilution | Clone | Source |
|---|---|---|---|---|
| CD31 Lin⁻ | BV421 | 1-100 | 390 | Biolegend |
| CD19 Lin⁻ | BV421 | 1-400 | 1A9 | BioLegend |
| Viability | AQUA | 1-1000 | HK1.4 | ThermoFisher |
| CD45 | BV605 | 1-150 | 30-F11 | Biolegend |
| CD11b | BV711 | 1-300 | 53-6.7 | Biolegend |
| CD3 | AF488 | 1-200 | GK1.5 | Biolegend |
| CD34 | PerCP-Cy5.5 | 1-100 | 6D5 | Biolegend |
| F4/80 | PE-Cy7 | 1-250 | BM8 | Biolegend |
| CD11b | AF700 | 1-400 | M1/70 | Biolegend |
| CD29 | APC-Cy7 | 1-100 | HMβ1-1 | Biolegend |

### Immunofluorescence Staining and Imaging

Dystrophin (rabbit anti-mouse monoclonal antibody, clone EPR21189, Abcam, dilution: 1:1000) was stained using tyramide signal amplification (TSA) method with Opal-650. Briefly, after blocking with bovine serum albumin, the first primary antibody was incubated at room temperature for 30 mins, followed by 30 mins of incubation with HRP polymer conjugated secondary antibody, and 10 mins of Opal-650. Slides were then counterstained with DAPI for 5 mins before being mounted using DAKO mounting medium. Imaging of the histological samples was performed on a Zeiss AxioObserver.Z2 and images were stitched on Zen Blue software.

### Cornea Tissue Processing and Flow Cytometry

Wounded corneas were collected from each experimental group (Saline vs. rSEA), 4-5 corneas were pooled for one “flow cytometry sample”. Cornea samples were processed similar to what is described in Ogawa, et al., with minor modification (68). Briefly, corneas in each group were digested in RPMI-1640 media containing 0.5 mg/mL Liberase TL (Sigma Aldrich)+0.2 mg/mL DNase I (Roche) for 45 min while gently rocking. Digested tissues were ground through 70 μm cell strainers and digestion stopped with FBS supplemented RPMI-1640. Cell suspensions were centrifuged, washed, and each cell pellet was resuspended in 200 μL 1×DPBS for staining and blocked with anti-mouse CD16/32 TruStain FcX (BioLegend) per manufacturer recommendations. The antibodies used were listed in Table 5 below.

TABLE 5
Mouse Cornea 4get IL4:GFP Pan-Immune

| Filter | Marker | Fluorophore | Dilution | Clone | Source |
|---|---|---|---|---|---|
| VL1 | Ly6g | Pacific Blue | 1-250 | 1A8 | Biolegend |
| VL2 | Ly6c | BV510 | 1-250 | HK1.4 | Biolegend |
| VL3 | CD45 | BV605 | 1-100 | 30-F11 | Biolegend |
| VL4 | CD8 | BV711 | 1-200 | 53-6.7 | Biolegend |
| BL1 | IL4-GFP | N/A | N/A | N/A | N/A |
| BL3 | CD11c | PerCP-Cy5.5 | 1-250 | N418 | Biolegend |
| GL1 | CD3 | PE | 1-150 | 17A2 | Biolegend |
| GL2 | Siglec F | PE-594 | 1-200 | E50-2440 | BD |
| GL4 | F4/80 | PE-Cy7 | 1-150 | EMR1 | Biolegend |
| RL1 | CD4 | APC | 1-250 | GK1.5 | Biolegend |
| RL2 | CD11b | AF700 | 1-250 | M1/70 | Biolegend |
| RL3 | Viability | eFluor780 | 1-1000 | N/A | Thermo |

### Cornea Model Immune Profile in Draining Lymph Nodes

Draining lymph nodes (submandibular lymph nodes) were collected and grinded through a 70 μm filter. Cells were collected after centrifugation and washing with 1×DPBS. Lymphocytes were stimulated for 4 hours with Cell Stimulation Cocktail, plus protein transport inhibitors (eBioscience), followed by staining of surface markers. After permeabilization and fixation of cells, cytokines IL-17A, and IFN-γ were stained for 4get mice and IL-17A, IL-4, and IFN-γ were stained for wild-type (WT) and GATA1 KO mice. The antibodies used are listed in Tables 6 and 7 below.

TABLE 6
Mouse Cornea ILN ICS Panel

| Filter | Marker | Fluorophore | Dilution | Clone | Source |
|---|---|---|---|---|---|
| VL1 | Thy1.2 | PacBlue | 1-500 | 53-2.1 | Biolegend |
| VL2 | CD45 | V500 | 1-100 | 30-F11 | BD |
| VL3 | NK1.1 | BV605 | 1-300 | PK136 | Biolegend |
| VL4 | CD8a | BV711 | 1-300 | 53-6.7 | Biolegend |
| BL1 | CD3 | AF488 | 1-200 | GK1.5 | Biolegend |
| BL3 | CD19 | PerCP-Cy8.5 | 1-200 | 6D5 | Biolegend |
| GL1 | IL-4 | PE | 1-200 | 11B11 | Biolegend |
| GL2 | γ6 | PE-594 | 1-300 | GL3 | Biolegend |
| GL4 | CD4 | PE-Cy7 | 1-300 | GK1.5 | Biolegend |
| RL1 | IFNγ | APC | 1-200 | XMG1.2 | Biolegend |
| RL2 | IL-17a | AF700 | 1-200 | TC11-18H10.1 | Biolegend |
| RL3 | Viability | eFluor780 | 1-1000 | N/A | Thermo |

TABLE 7
Monse Cornea ILN 4get IL4:GFP Modified ICS

| Filter | Marker | Fluorophore | Dilution | Clone | Source |
|---|---|---|---|---|---|
| VL1 | Thy1.2 | PacBlue | 1-500 | 53-2.1 | Biolegend |
| VL2 | CD45 | V500 | 1-100 | 30-F11 | BD |
| VL3 | NK1.1 | BV605 | 1-300 | PK136 | Biolegend |
| VL4 | CD8a | BV711 | 1300 | 53-6.7 | Biolegend |
| BL1 | CD3 | AF488 | N/A | N/A | N/A |
| BL3 | CD19 | PerCP-Cy8.5 | 1-200 | 6D5 | Biolegend |
| GL1 | IL-4 | PE | 1-200 | 11B11 | Biolegend |
| GL2 | γ6 | PE-594 | 1-300 | GL3 | Biolegend |
| GL4 | CD4 | PE-Cy7 | 1-300 | GK1.5 | Biolegend |
| RL1 | IFNγ | APC | 1-200 | XMG1.2 | Biolegend |
| RL2 | IL-17a | AF700 | 1-200 | TC11-18H10.1 | Biolegend |
| RL3 | Viability | eFluor780 | 1-1000 | N/A | Thermo |

### Cornea Immunostaining

Dissected corneas were fixed in 100% methanol at −20° C. for 30 min, and permeabilized with PBS containing 0.25% Triton-X (PBST). The cornea samples were blocked with 1% goat serum+1% BSA in PBST for 30 mins and stained with rabbit anti-mouse αSMA (Abcam) overnight at 4° C. Following washing with PBST, corneas were stained with goat anti-rabbit 633 for 2 hrs at room temperature, and mounted flat in SlowFade Diamond Antifade Mountant (Thermo Fisher Scientific). Zeiss Apotome microscope was used for fluorescent imaging.

### Statistics

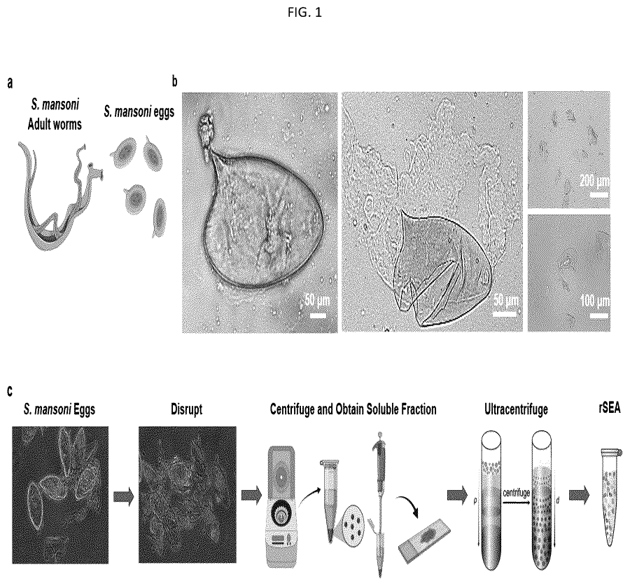

Data points for all in vivo experiments are biological replicates and were not randomly assigned. Investigators were not formally blinded to separation during experiments and outcome assessment, except for histological assessment for scoring. All experiments were independently replicated with similar results and trends at least twice, except sorted T cell and macrophages NanoString results in FIGS. 1h and 1m as previously noted, which were each performed once. No data were excluded from the study. No formal statistical methods were used to determine sample size and differences of intra-group variances; however, sample sizes were determined by previous experiences with injury models and immunotherapy treatments and their respective previous power analyses in previous publications within the laboratory (4, 57, 69). All statistical differences were determined using GraphPad Prism (version 9.2.0 for Windows, GraphPad Software), excluding NanoString Codeset results, which were analyzed using nSolver Advanced Analysis Software. All other data was analyzed using two-way ANOVA with Sidak's multiple comparisons for experiments with two or more independent factors (KO mouse models vs. WT, and gene expression of multiple genes) wherein experimental group conditions were arranged by column and mouse strain or gene of interest were listed as row factors. Ordinary one-way ANOVA with Tukey's multiple comparisons was used for experiments with three or more experimental groups, comparing one factor each. In all other cases, unpaired, two-tailed Student's t-tests were used for single factor conditions wherein saline treatment controls were compared only to rSEA treated groups. NanoString differential expression results for sorted CD3⁺ T cells and F4/80^{Hi+} macrophages were analyzed using the manufacturer supplied nSolver Advanced Analysis Software (version 4.0.70, NanoString Technologies, inc.) according to manufacturer guidelines and recommendations. NanoString differential gene expression analysis was performed using the Advanced Analysis with an analysis threshold of 20 counts (minimum) per gene probe was used and the automated software selected the top 16 reference genes for analysis of the sorted macrophages and the top 10 reference genes were selected by the software for the sorted T cell analysis. For NanoString analysis, False Discovery rate-adjusted P-values were determined for each gene by applying the Benjamini-Yekutieli method.

### Flow Cytometry Analysis

All flow cytometry data was visualized, analyzed, and gated using FlowJo (version 10.7.1 for Windows, BD Life Sciences). Gating for positive populations utilized fluorescence-minus-one controls, and all reported populations for these studies are from events on-scale, singlets (using the diagonal gating of FSC-Height vs. FSC-Area cells, live (negative for amine-reactive dye stain), CD45-positive events, with negative/positive gating as shown in FIG. 17. To ensure the integrity of the reported results, populations were backgated and screened and quality controlled for unusual characteristics (e.g., CD3⁺ sub-populations expressing CD19 were omitted from evaluation).

### Example 2

Development of a Type 2 Immunotherapy from S. mansoni Eggs Antigens

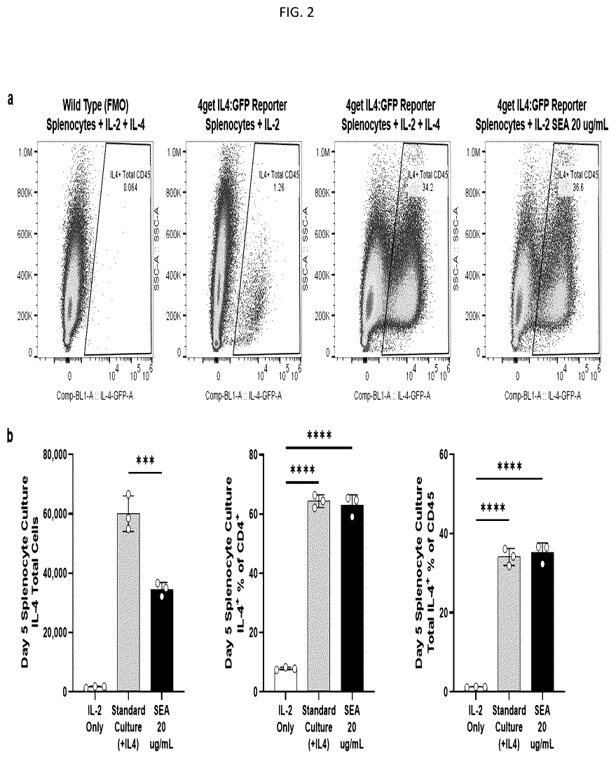

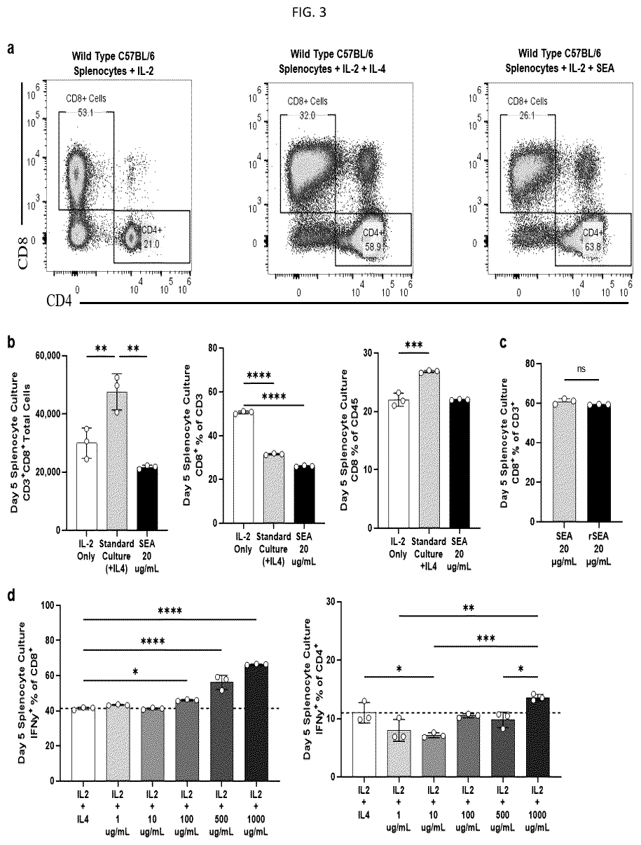

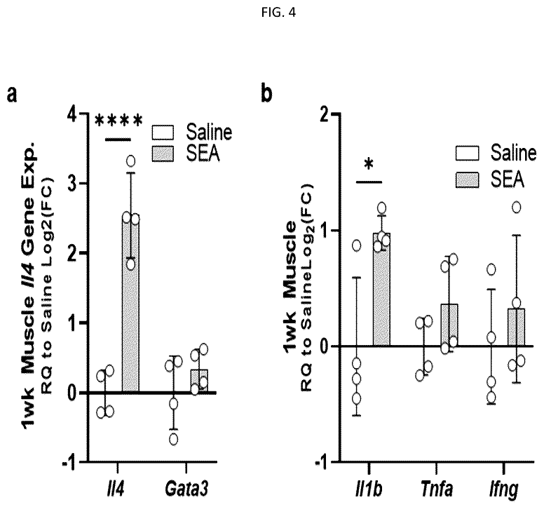

The soluble egg antigen (SEA) extract from S. mansoni is composed of hundreds to thousands of proteins, glycoproteins, and lipids depending on the extraction protocol, and is well recognized in its ability to stimulate a type 2 immune response (31-34). It was first sought to determine if SEA could efficiently stimulate a type 2 immune response that would promote tissue repair without deleterious inflammation or fibrosis. S. mansoni eggs isolated from infected mice were mechanically disrupted and ultracentrifuged to remove insoluble components to isolate SEA as described by Boros, et al. (FIG. 1) (28). The immune response to SEA was first evaluated using an in vitro screen with splenocytes from C57BL/6 wild type and IL-4-eGFP reporter (4get) mice. Addition of SEA to the splenocyte culture resulted in increased expression of IL-4-eGFP by CD4 T cells comparable to IL-4 (FIG. 2), however there was also a modest increase in CD8⁺ T cells expressing IFN-γ as measured by cytokine staining in C57BL/6 splenocyte cultures (FIG. 3). To further test in a wound healing model, a volumetric muscle loss wound (VML) in female mice was created to evaluate the repair capacity of SEA. Application of a single dose of SEA to the VML at the time of injury resulted in a significant increase in Il4 gene expression in the muscle tissue 1-week post-surgery (FIG. 4A). However, similar to the in vitro studies, there was also a significant increase in inflammatory cytokine expression found in Ifng, Illb, and Tnfa in the muscle tissue with SEA treatment compared to saline treated control injury (FIG. 4B).

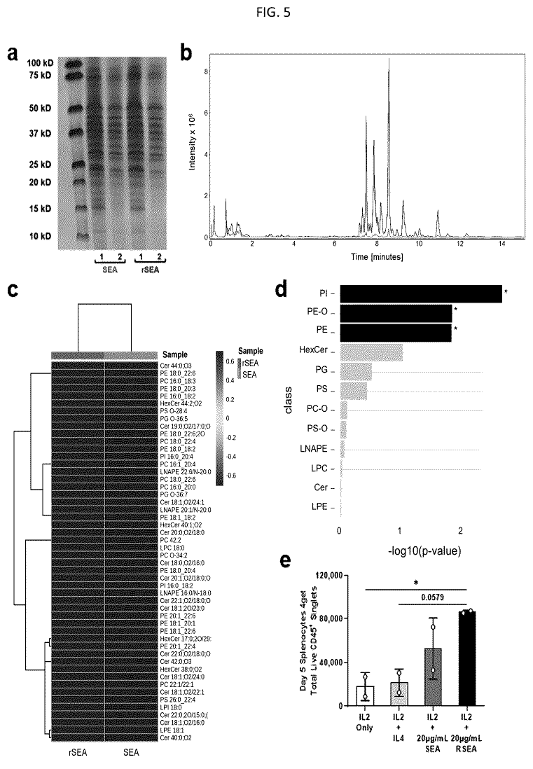

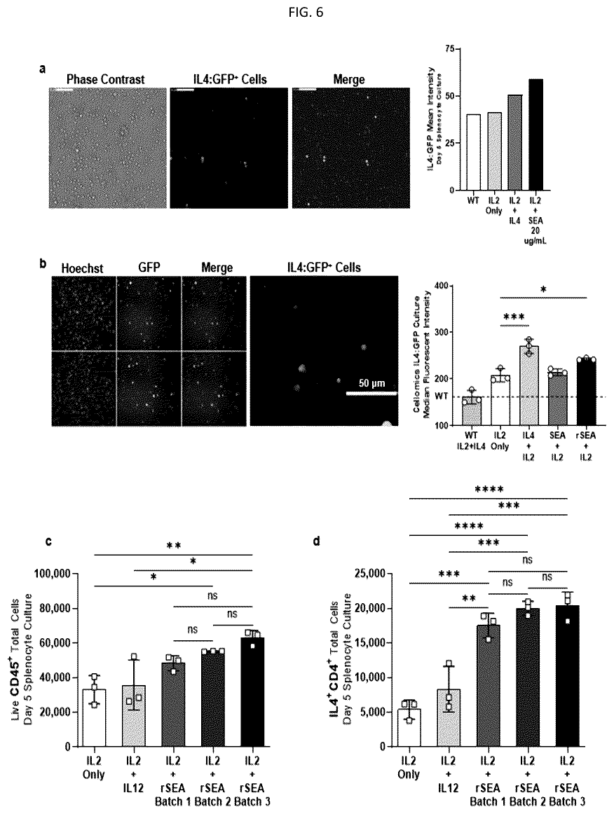

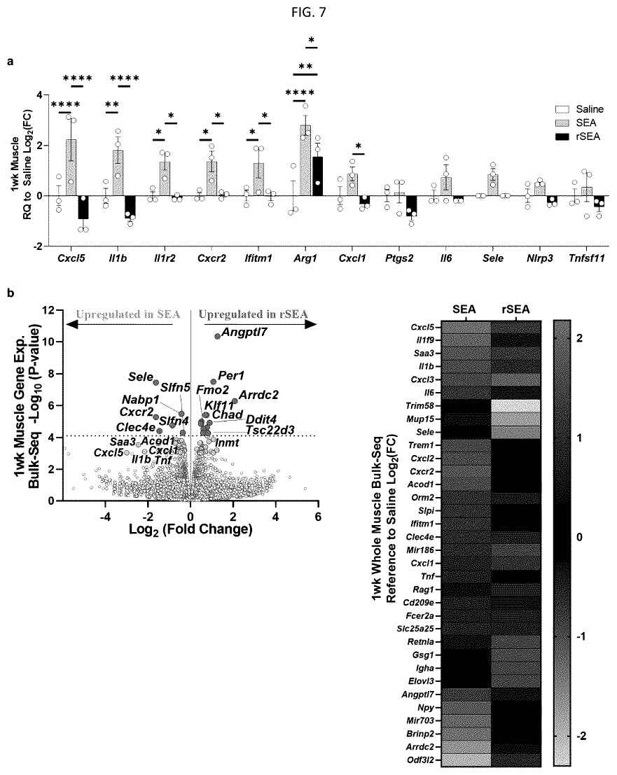

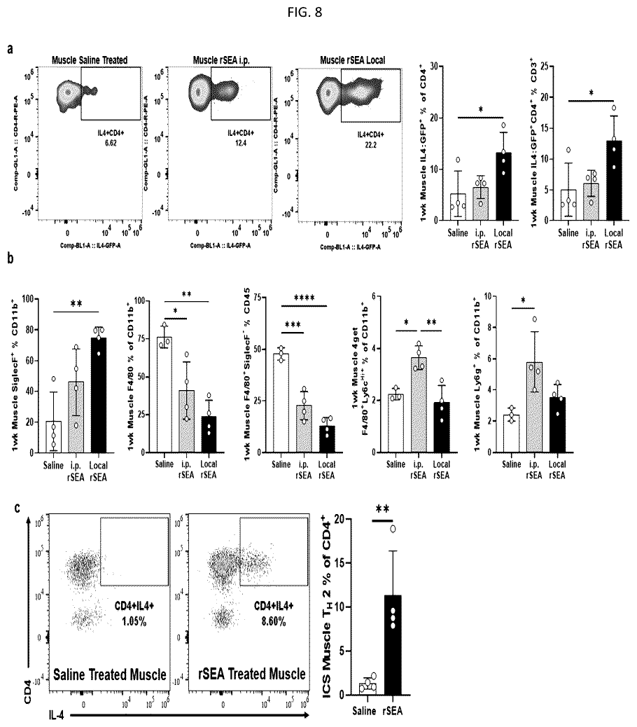

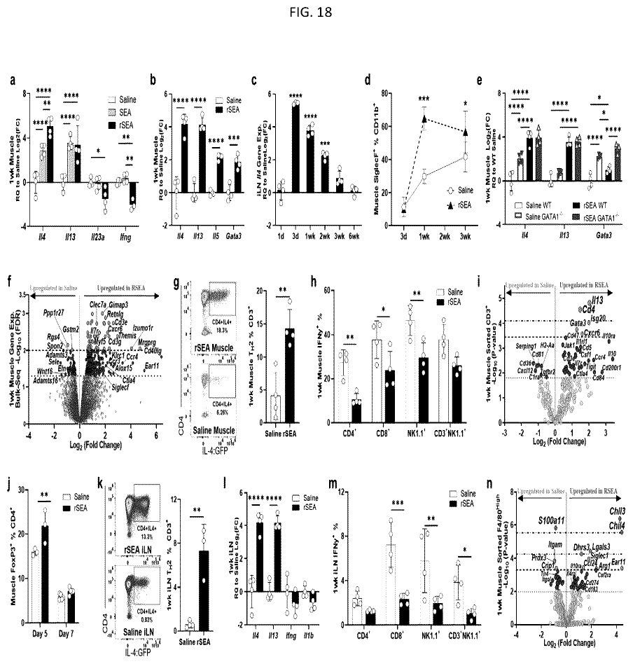

To determine if SEA could be formulated to stimulate a dedicated type 2 regenerative immune response without increased interferon-dependent pathological inflammation, the SEA isolation was modified to extract specific layers of the ultracentrifuged egg supernatant. In particular, the SEA was further purified by isolating the lower density soluble fraction and combined it with the lipid portion (See, FIG. 22), as recent lipidomic research reported that S. mansoni worms and eggs contain pro-resolving mediators which can promote wound healing (35, 36). Protein gel electrophoresis did not reveal distinct changes in SEA protein composition with the modified isolation protocol (FIG. 5A). In contrast, lipid analysis via liquid chromatography mass spectrometry (LC-MS) demonstrated that the modified isolation protocol resulted in a large increase in lipid content (FIG. 5B). Further lipidomic analysis revealed changes in the lipid class and individual lipid levels between the standard and modified SEA formulations at both the lipid class and individual lipid levels (FIG. 5C-FIG. 5D). The in vitro 4get splenocyte screening assay confirmed IL-4 stimulation along with cell proliferation with the modified SEA formulation (FIG. 5E, FIGS. 6A-6B) that was consistently generated from multiple batches (FIGS. 6C-6D). Application of the modified SEA formulation to the VML injury model resulted in a significant increase in Il4 gene expression in the muscle tissue without a concomitant increase in Ifng and Illb expression (FIG. 18A, FIG. 7A). Instead, expression of these inflammatory genes decreased with the modified SEA treatment compared to standard SEA and saline treated injuries one-week post-surgery. Bulk RNA sequencing confirmed significant differences between classical SEA and the modified SEA formulation (FIG. 7B). This formulation was then used for all subsequent studies and is referred to as regenerative SEA (rSEA). The different delivery modalities for the rSEA formulation were evaluated by comparing local and systemic (intraperitoneal, IP) injection. Both local delivery in the VML wound and systemic IP injection of rSEA increased IL-4 expression in the muscle tissue one week after injury (FIGS. 8A-8B). However, further examination of specific immune subsets found that local rSEA treatment significantly increased both IL-4 expressing T cells and eosinophils more than double compared to systemic delivery (FIG. 8B) so local treatment was used for all subsequent studies.

rSEA Treatment Increases IL4-Expressing Eosinophils, T_{H}2 T Cells, and Regulatory T Cells in a Volumetric Muscle Loss Injury

Since type 2 immune signals are critical for muscle injury repair, the kinetics and cellular sources of IL-4 were evaluated after rSEA treatment. A single treatment of rSEA at the time of injury increased Il4 gene expression in the muscle tissue 20- to 30-fold, relative to saline treated muscles, at one-week post-surgery (FIG. 18B). Treatment with rSEA also increased expression of other type 2 associated genes including Il5, Il13, and Gata3 (FIG. 18B). Furthermore, the Il4 gene expression in the draining inguinal lymph nodes increased, peaking at 3 to 7 days post-treatment and returning to the level of saline treated controls after 3-weeks (FIG. 18c), suggesting that a single rSEA treatment was able to modulate the tissue immune environment for weeks.

Eosinophils are well recognized as a source of IL-4 after muscle injury the changes in these cell types after rSEA treatment was first evaluated using multiparametric flow cytometry. Eosinophils (CD11b⁺SiglecF⁺SSC^{Hi}), which depend on type 2 immune cytokines and chemokines such as IL-5 and eotaxin, were the most abundant CD11b⁺ cell population in the muscle 1-week post rSEA treatment, producing an 11-fold increase in the number of cells that remained significantly higher compared to saline treated controls at 3-weeks post-treatment (FIG. 18D). However, Il4 gene expression in the muscle tissue still increased in ΔdblGATA (GATA1 knock-out) mice, which lack eosinophils, suggesting that rSEA is stimulating other cell types in the muscle wound (FIG. 18E).

At 1-week post-injury, bulk-RNA sequencing was performed on rSEA treated skeletal muscle and found a wide variety of differential gene expression relative to the saline treated muscles (FIG. 18F). Bulk-sequencing analysis identified 403 differentially expressed genes (330 upregulated, 73 downregulated) using the semi-conservative EdgeR analysis, with the top upregulated genes associated with type 2 immunity, natural killer cell activity, T cell activation, antigen processing, and the downregulation of multiple inflammatory genes (Table 8).

TABLE 8

| Gene Name | RSEA LOG2(FC) | EDGE R FDR | Gene Name | RSEA LOG2(FC) | EDGE R FDR |
|---|---|---|---|---|---|
| Gimap3 | 1.774081225 | 0.001014808 | H2-T23 | 0.610937854 | 0.00945388 |
| Atp1b4 | 0.968509205 | 0.001035301 | Acap1 | 1.224430208 | 0.009713101 |
| Retnlg | 2.027560396 | 0.001324974 | Gstm2 | −0.586071007 | 0.009713101 |
| Gatm | 0.964943966 | 0.001616492 | C [symbol] ta | 0.810464103 | 0.00995403 |
| Icos | 1.679668492 | 0.001631082 | ftk | 1.481613368 | 0.010086007 |
| Cd3e | 2.629457564 | 0.001631082 | Shisal1 | 0.823129696 | 0.010086007 |
| Iqsec3 | 0.66606271 | 0.001631082 | Molfi | 0.879013473 | 0.010086007 |
| Tm5sf1 | 0.639572302 | 0.001631082 | H2-Q5 | 0.9646686 | 0.010086007 |
| Cd6 | 2.261516917 | 0.001631082 | Spon2 | −1.277600493 | 0.010086007 |
| Ms4a4b | 1.939024737 | 0.001900998 | Trbc1 | 1.820173232 | 0.010479107 |
| Ciec7a | 1.336884384 | 0.001900998 | AW112010 | 0.81226642 | 0.010479107 |
| Cxcr6 | 2.161408089 | 0.003257059 | Sla2 | 2.004608338 | 0.010892934 |
| Vash2 | 0.631238466 | 0.003297848 | Apobec1 | 0.845130629 | 0.010892934 |
| Lck | 1.544619208 | 0.003340316 | Rab44 | 1.279718226 | 0.010892934 |
| Traf3ip3 | 0.651254 [symbol] | 0.004170501 | Zap70 | 0.966377907 | 0.010892934 |
| Card11 | 1.149250752 | 0.004170501 | Skap1 | 1.83346263 | 0.010892934 |
| Zdbf2 | 0.757353056 | 0.004170501 | Slamf8 | 1.126284884 | 0.010892934 |
| Cd3g | 2.287459767 | 0.004170501 | Myl4 | 0.601788015 | 0.011040333 |
| Il2rb | 1.371027256 | 0.004170501 | Mpeg1 | 0.770643045 | 0.011160855 |
| Cd5 | 2.006777738 | 0.004170501 | Rgs4 | −0.711988522 | 0.011610116 |
| Themis | 2.963198744 | 0.004170501 | [symbol] zf3 | 1.407876452 | 0.011664359 |
| Stra6l | 1.425453694 | 0.004170501 | Mrch2a | 1.989042933 | 0.011664359 |
| Il7r | 1.187737935 | 0.004170501 | Cysitr2 | 2.373203681 | 0.011683291 |
| Fmo1 | 0.638195278 | 0.004170501 | Mymx | 0.767737528 | 0.011683291 |
| Myf5 | 0.973625834 | 0.00568838 | Pglyrp2 | 1.775772842 | 0.01177976 |
| Ppfia4 | 0.745441167 | 0.00568838 | Pcbd1 | 0.950335204 | 0.011895076 |
| Cd83 | 0.82843717 | 0.00568838 | Cd300c2 | 0.635282072 | 0.012520707 |
| Rasal3 | 1.016682102 | 0.005856345 | Mymk | 0.762518825 | 0.012520707 |
| Mycl | 0.798236513 | 0.006919635 | Itgal | 0.938468557 | 0.012702262 |
| Pppfr27 | −0.932630095 | 0.007167036 | Lair1 | 0.826924818 | 0.012702262 |
| Slamf7 | 0.880797602 | 0.007978069 | Napsa | 0.876640816 | 0.013824616 |
| Tmem37 | 0.65915643 | 0.007978069 | Flnb | −0.532382412 | 0.013824616 |
| Cyp4f18 | 1.316153996 | 0.008469018 | Sell | 1.199061719 | 0.013917042 |
| Alf1l | 0.761876136 | 0.008469018 | Arbgap25 | 0.724774454 | 0.013984896 |
| Kira13-ps | 2.313017578 | 0.008862624 | Gimap7 | 2.079902981 | 0.014144676 |
| Ltb | 1.407502929 | 0.008862624 | Cxcr3 | 1.194714561 | 0.014220666 |
| fzumo1r | 4.042164981 | 0.008862624 | H2-Q4 | 0.700165392 | 0.014220666 |
| Mrgprg | 4.722399407 | 0.008892888 | Pax7 | 0.619742274 | 0.014220666 |
| Cd3d | 1.912507389 | 0.00907744 | Lat2 | 0.719892138 | 0.014220666 |
| Irf8 | 0.74119657 | 0.00945388 | Gaint5 | 0.515951376 | 0.014249946 |
| Tnfrsf18 | 1.47349947 | 0.01449763 | Cdkn1c | 0.499689081 | 0.01782567 |
| Ptprc | 0.730409657 | 0.014942951 | Cd300a | 0.747003552 | 0.01782567 |
| Cd40lg | 6.394679562 | 0.014942951 | Lgais3 | 0.809897439 | 0.018296341 |
| Tceal3 | 0.902042352 | 0.014942951 | Trim12c | 0.645426162 | 0.016337938 |
| Pnmal2 | 0.816945012 | 0.014942951 | Gm3830 | 0.916942956 | 0.018482818 |
| Tcf7 | 0.790346612 | 0.015447083 | Gbp3 | 0.846697324 | 0.018482818 |
| AU020206 | 0.726493508 | 0.015456061 | Mapk8ip1 | 0.617150874 | 0.018482818 |
| Adamts3 | −0.652167887 | 0.015924902 | P2ry14 | 0.986061032 | 0.020522596 |
| Ninl | 0.839606866 | 0.015924902 | Pde3a | −0.462021669 | 0.020522596 |
| Rhoh | 0.835490551 | 0.015947606 | Gm14635 | 1.27629562 | 0.020884486 |
| Gzmb | 1.632963097 | 0.01647679 | 2310043L19Rik | 1.532807489 | 0.020914086 |
| F5 | 1.479467736 | 0.01647679 | Ly86 | 0.701639678 | 0.021886266 |
| Ms4a7 | 0.659934383 | 0.01647679 | Cd2 | 1.116446619 | 0.021886266 |
| Gpr66 | 0.868091118 | 0.01647679 | Ccr3 | 2.176071664 | 0.021886266 |
| Fgfr11 | 0.687827093 | 0.01647679 | Otog | 1.038769913 | 0.021886266 |
| Kcnj2 | −0.677460472 | 0.01647679 | Adap1 | 0.774366267 | 0.022281282 |
| Slc15a3 | 0.696268435 | 0.016531819 | Pgm5 | 0.433422664 | 0.022307191 |
| Neurl3 | 0.890350257 | 0.016531819 | Slfn1 | 1.102949565 | 0.022482658 |
| Chil3 | 1.729960162 | 0.016531819 | Iflt1 | 0.83221217 | 0.022482658 |
| Hexb | 0.476583455 | 0.016531819 | Sytl2 | 0.42735373 | 0.022482658 |
| Ubash3a | 1.667416719 | 0.016839465 | Slc9a3r2 | −0.451757442 | 0.022482658 |
| Trac | 1.613445515 | 0.016839465 | A630023P12Rik | 4.16812957 | 0.022482658 |
| Siglech | 1.11860435 | 0.016839465 | Nuak1 | −0.408563432 | 0.022482658 |
| Septin1 | 0.756597008 | 0.016839465 | Trbc2 | 1.811767895 | 0.022482658 |
| Gm11243 | 1.600647165 | 0.016839465 | Patj | 0.703552208 | 0.022482658 |
| Ccl5 | 1.002775988 | 0.016839465 | Ccr4 | 3.352362266 | 0.022482658 |
| Rtkn2 | 0.813259595 | 0.016839465 | Vcam1 | 0.654389692 | 0.022482658 |
| Bcl11b | 1.520176893 | 0.016839465 | Cytip | 0.819007246 | 0.022482658 |
| Fgfr4 | 0.791767117 | 0.016839465 | Sele | −1.49660031 | 0.022482658 |
| Cdk5r1 | 0.683872361 | 0.016901209 | H2-Aa | 0.872081618 | 0.022746521 |
| Loxl2 | −0.534472869 | 0.016972871 | Zfp185 | −0.699363114 | 0.02277294 |
| Irf7 | 0.614684325 | 0.016972871 | Gdf15 | 1.395769212 | 0.02277294 |
| Ngk7 | 1.089445228 | 0.016972871 | Gd52 | 0.863732181 | 0.023517717 |
| Ctss | 0.894274205 | 0.016972871 | Prune2 | 0.601806369 | 0.023553097 |
| Itgb7 | 0.866366192 | 0.016972871 | Foxp3 | 1.552171236 | 0.023991236 |
| Jpt1 | 0.440059004 | 0.016972871 | Plekhh1 | 0.749985754 | 0.023991236 |
| Kirc1 | 2.407646291 | 0.017064327 | Rcan1 | −0.445639927 | 0.023993692 |
| Erbb3 | 0.426132693 | 0.017731346 | Olfr1372-ps1 | 1.413258489 | 0.024037961 |
| Slfn2 | 0.695843798 | 0.017761234 | Sh2d2a | 1.343267424 | 0.024037961 |
| Cracd | −0.932450088 | 0.01782567 | lpcef1 | 1.086447627 | 0.024260598 |
| Myod1 | 0.586967045 | 0.01782567 | id2 | 0.779833034 | 0.024260598 |
| Lpcat2 | 0.841645769 | 0.024260598 | P2ry10 | 0.963606571 | 0.030819143 |
| San30 | 0.672006761 | 0.024260598 | Parm1 | −0.479015964 | 0.030819143 |
| Slamf6 | 1.279177657 | 0.024260598 | Wnt2b | −0.64172016 | 0.031402135 |
| Gbp8 | 1.198103852 | 0.024390679 | Eln | −0.954410711 | 0.031644896 |
| Alox15 | 2.456866703 | 0.024390679 | Atp6v0d2 | 1.074245627 | 0.031704486 |
| H2-M2 | 2.74241885 | 0.024390679 | Ccdc141 | 0.564170964 | 0.031711884 |
| Fcgr4 | 0.841491825 | 0.024390679 | Htr2a | −0.706011003 | 0.031812597 |
| Mmel1 | 1.080338641 | 0.024469362 | Ikzf1 | 0.642086496 | 0.031943659 |
| Fgd5 | −0.469197654 | 0.024679312 | Tmem86a | 0.56022246 | 0.031943659 |
| Sigiecg | 1.081476208 | 0.024778719 | H2-DMb1 | 0.695076314 | 0.031943659 |
| Cebpe | 1.609893133 | 0.024922707 | Fbn2 | −0.828907818 | 0.031943659 |
| Resf1 | 0.520722269 | 0.02636228 | H2-Ab1 | 0.841452475 | 0.031943659 |
| Enho | 0.637747064 | 0.02636228 | Cyria | 0.376624396 | 0.031996056 |
| Mamstr | 0.497002253 | 0.025395155 | Gzma | 1.213832426 | 0.032659141 |
| H2-Eb1 | 0.646753911 | 0.025395155 | Unc13d | 0.791582052 | 0.032659141 |
| Itgax | 0.933921866 | 0.025395155 | Jaml | 0.944407219 | 0.032659141 |
| R3hdml | 1.075418321 | 0.025967065 | Sla | 0.946216256 | 0.032659141 |
| Ctla4 | 2.057700843 | 0.026686783 | Ccdc134 | 0.467089519 | 0.032659141 |
| Coro1a | 0.626072201 | 0.027030909 | H2-DMa | 0.713480165 | 0.032659141 |
| Tnnt2 | 0.669893519 | 0.027063831 | Misd12 | 0.628531101 | 0.032659141 |
| Klrk1 | 1.106366517 | 0.027106533 | Gpr55 | 1.627874478 | 0.032659141 |
| Npnt | 0.423662396 | 0.027193933 | Pcdh12 | −0.530553348 | 0.032659141 |
| Cd22 | 0.901981731 | 0.027452862 | ligp1 | 0.673710706 | 0.032725333 |
| Plcb2 | 0.683624616 | 0.027470048 | Pcp4l1 | −0.509201304 | 0.032785894 |
| Gaint16 | −0.627205659 | 0.027470048 | Kcnma1 | −0.639673641 | 0.033025648 |
| Tlr12 | 1.261613232 | 0.027470048 | Atf3 | 0.54748947 | 0.033459288 |
| Gm49654 | 1.182247466 | 0.027561142 | Stmn4 | −0.924092492 | 0.033552969 |
| Pd2rn3 | −0.450999491 | 0.027612909 | Fndc3c1 | 0.709038093 | 0.033641773 |
| Ptprcap | 0.995839479 | 0.027784426 | Tlr1 | 0.687738362 | 0.033917164 |
| Gm47992 | 2.824835237 | 0.027784426 | Ptgds | 1.339613098 | 0.033917164 |
| Il18rap | 0.872373966 | 0.027936763 | Gm45867 | 1.081683115 | 0.033917164 |
| Myog | 0.681487392 | 0.028020689 | Gm4951 | 0.759214624 | 0.033917164 |
| Bcat1 | −0.719509862 | 0.028644926 | Flt3 | 0.648262906 | 0.033917164 |
| Gm8369 | 1.532397581 | 0.028944016 | Tubb4b | −0.364677636 | 0.033917164 |
| Tbc1d8 | 0.396174299 | 0.029412281 | Adamts4 | −0.689364541 | 0.033917164 |
| Lat | 1.781419338 | 0.030519305 | Cxcr4 | 0.650796114 | 0.033917164 |
| Palm3 | 0.737096113 | 0.030519305 | Arntl | −0.55020371 | 0.033947996 |
| Ddx60 | 0.639162327 | 0.030601259 | Hk3 | 0.680138919 | 0.034098072 |
| Cd300lf | 1.031116269 | 0.030601259 | Rab11fip4 | 0.885421817 | 0.034098072 |
| Klrg1 | 1.871663116 | 0.030601259 | Treml2 | 1.132409903 | 0.034312196 |
| Hspa12b | −0.384728096 | 0.03076527 | Rsad2 | 0.730982347 | 0.034365881 |
| Epsti1 | 0.705626268 | 0.034468208 | Coro2a | 0.835879418 | 0.037095935 |
| Lax1 | 0.644662067 | 0.034468208 | Abcg1 | 0.707529309 | 0.037095935 |
| Cpe | −0.676864761 | 0.034468208 | Samsn1 | 0.871336494 | 0.037095935 |
| Lacc1 | 0.649664346 | 0.034468208 | Nr1d1 | −0.685606911 | 0.037095935 |
| Gpr174 | 1.328695861 | 0.034468208 | Blnk | 0.731914368 | 0.037095935 |
| Slc44a4 | 1.244062096 | 0.034468208 | Ms4a14 | 0.563406618 | 0.037178737 |
| Tbx4 | 0.495827782 | 0.034468208 | Fzd9 | −0.568660747 | 0.037178737 |
| Tap1 | 0.621941062 | 0.034468208 | F2rl1 | −0.904869668 | 0.037178737 |
| Myo1f | 0.585952369 | 0.034686363 | Marchf1 | 0.524306446 | 0.037178737 |
| Il18r1 | 1.196470761 | 0.034638793 | Sat1 | 0.503616149 | 0.037178737 |
| Ctsw | 1.244757716 | 0.035012634 | Tifab | 0.620463746 | 0.037178737 |
| Arhgap42 | −0.344640414 | 0.035012634 | Cd84 | 0.750274677 | 0.037353396 |
| Rassf7 | 0.804695674 | 0.035012634 | Tnc | −0.831608758 | 0.037805842 |
| Gstm1 | 0.380854387 | 0.036212128 | Psmb9 | 0.636961799 | 0.037842163 |
| StBsia1 | 1.437596983 | 0.036212128 | 2410006H16Rik | 0.446597775 | 0.037842163 |
| Gamt | 0.400409236 | 0.036212128 | Trim25 | 0.314681693 | 0.037862711 |
| Slc2a9 | 0.833426321 | 0.036212128 | Aph1c | 0.800620959 | 0.036104412 |
| Nlrp1b | 0.790719529 | 0.035864933 | Hvcn1 | 0.597481125 | 0.036124734 |
| Rasgrp1 | 0.739165507 | 0.035864933 | P4ha2 | −0.435577498 | 0.03834952 |
| Eomes | 1.455063296 | 0.035864933 | Celsr3 | 1.143381456 | 0.03834952 |
| Il1a | 1.886532679 | 0.035864933 | Sdsl | 1.011682242 | 0.03834952 |
| A430093F15Rik | 1.876909536 | 0.036035889 | Aldh1l2 | −0.62840189 | 0.03834952 |
| Mirt1 | 1.628082956 | 0.036035889 | Grin2a | 1.509816526 | 0.03861339 |
| St3gal2 | −0.36949867 | 0.036035889 | Ifi208 | 0.886325243 | 0.038737167 |
| Tapbpl | 0.450679667 | 0.036035889 | Klrd1 | 1.3675707 | 0.038737167 |
| Cybb | 0.634826894 | 0.036035889 | Arhgap45 | 0.534820686 | 0.038737167 |
| Tmem71 | 0.889817308 | 0.036035889 | Sit1 | 2.292654702 | 0.038737167 |
| Rnase2a | 6.069616946 | 0.036035889 | Il10ra | 0.530499534 | 0.038737167 |
| Fndc3b | −0.546850861 | 0.036244419 | Igfbp2 | 1.051632314 | 0.038737167 |
| Gbp7 | 0.473441494 | 0.036426896 | Etr3b | −0.639582132 | 0.038737167 |
| AB124611 | 0.68282944 | 0.036623256 | Cd200r4 | 0.823100788 | 0.038894303 |
| Has3 | −0.834611496 | 0.036773038 | Rasgef1b | 0.633532356 | 0.039067339 |
| Dym | −0.409421834 | 0.036834063 | Pycr1 | −0.478254643 | 0.039205288 |
| Trit1 | 0.409532286 | 0.036834063 | Vstm2a | 1.602975248 | 0.040011216 |
| Dll1 | 0.549246697 | 0.036834063 | Parvg | 0.795249187 | 0.040421056 |
| Sic8a1 | −0.364611677 | 0.036834063 | Crim1 | −0.39210798 | 0.040885426 |
| Piezo2 | −0.476712587 | 0.036834063 | Xcr1 | 1.137449203 | 0.041516018 |
| Togaram2 | −0.639179347 | 0.036879592 | Ifi209 | 0.632553477 | 0.041569611 |
| Smim6 | 1.717852717 | 0.036910898 | Pamr1 | −0.391608223 | 0.041569611 |
| Tap2 | 0.408039862 | 0.036910698 | Igsf6 | 0.714111169 | 0.041569611 |
| H2-T24 | 0.461962001 | 0.037095035 | Dna2 | 0.523613196 | 0.041569611 |
| Coro2a | 0.835879418 | 0.037095935 | Arhgap9 | 0.712603675 | 0.041569611 |
| Abcg1 | 0.707629309 | 0.037095935 | Creb3l1 | −0.865616922 | 0.041569611 |
| Samsn1 | 0.871335494 | 0.037095935 | Col26a1 | 0.510766934 | 0.041569611 |
| Nr1d1 | −0.685606911 | 0.037095935 | Ogdhl | 0.669316353 | 0.041569611 |
| Blnk | 0.731914358 | 0.037095935 | Ap1s3 | 1.433898703 | 0.041569611 |
| Ms4a14 | 0.553406618 | 0.037178737 | Fcho1 | 1.028392561 | 0.041569611 |
| Fzd9 | −0.568880747 | 0.037178737 | Stk17b | 0.410569622 | 0.041687058 |
| F2rl1 | −0.904869668 | 0.037178737 | Jcad | −0.485486249 | 0.042172923 |
| Marchf1 | 0.524305446 | 0.037178737 | Gm45512 | 0.735982419 | 0.042418902 |
| Sat1 | 0.503616149 | 0.037178737 | Ifit2 | 0.642423464 | 0.042777243 |
| Tifab | 0.620463746 | 0.037178737 | Lrrc17 | −0.467419675 | 0.042777243 |
| Cd84 | 0.750274677 | 0.037363396 | Krt8 | 0.905314267 | 0.042777243 |
| Tnc | −0.631608756 | 0.037805842 | Slc38a1 | 0.570037105 | 0.042777243 |
| Psmb9 | 0.638961799 | 0.037842163 | Mypop | −0.526028724 | 0.042777243 |
| 2410006H16Rik | 0.446597776 | 0.037842163 | Kdm3a | 0.306834967 | 0.042777243 |
| Trim25 | 0.314681893 | 0.037882711 | Ly9 | 0.679601423 | 0.042777243 |
| Aph1c | 0.800620959 | 0.038104412 | H2-Q7 | 0.906950083 | 0.042777243 |
| Hvcn1 | 0.597481125 | 0.038124734 | Cd82 | 0.427879879 | 0.042777243 |
| P4ha2 | −0.436577498 | 0.03834952 | Pgtyrp1 | 0.662597139 | 0.042777243 |
| Ceisr3 | 1.143381456 | 0.03834952 | Trim30a | 0.605194863 | 0.043036209 |
| Sdsl | 1.011682242 | 0.03834952 | Rgs2 | 0.493291454 | 0.043347892 |
| Aldh1l2 | −0.62840189 | 0.03834952 | Oas3 | 0.650472612 | 0.043347892 |
| Grin2a | 1.609818526 | 0.03861339 | Ttc39a | 0.655496121 | 0.043347892 |
| If1208 | 0.886325243 | 0.038737167 | Hsh2d | 1.119041471 | 0.043347892 |
| Kird1 | 1.3672707 | 0.038737167 | Gal | 2.533418974 | 0.043436345 |
| Arhgap45 | 0.634820686 | 0.038737167 | Gm8113 | 2.425659458 | 0.043436345 |
| Sit1 | 2.292664702 | 0.038737167 | Manf | −0.443449248 | 0.043701864 |
| Il10ra | 0.530499534 | 0.038737167 | Parp10 | 0.369145034 | 0.043701864 |
| Igfbp2 | 1.051632314 | 0.038737167 | Kcnh2 | 0.720406589 | 0.043846894 |
| Efr3b | −0.639562132 | 0.038737167 | Map7d3 | 0.997716956 | 0.044218604 |
| Cd200r4 | 0.823100788 | 0.38894203 | B3gnt5 | 0.884014731 | 0.044218604 |
| Rasgef1b | 0.533532356 | 0.039057339 | Siglect | 1.642229776 | 0.044218604 |
| Pycr1 | −0.476254643 | 0.039205288 | H2-K1 | 0.626893736 | 0.044218604 |
| Vstm2a | 1.602975248 | 0.040011216 | Sh2d1b1 | 0.921322008 | 0.044218604 |
| Parvg | 0.796249187 | 0.040421056 | Cmah | −0.627998276 | 0.044218604 |
| Crim1 | −0.39210798 | 0.040885428 | Jam3 | 0.307453607 | 0.044218604 |
| Xcr1 | 1.137449203 | 0.041815018 | Arhgef15 | −0.471622801 | 0.044218604 |
| Ifl209 | 0.632553477 | 0.041569611 | Al504432 | 0.549653117 | 0.044328397 |
| Pamr1 | −0.391606223 | 0.041569611 | Tm4sf1 | −0.339333547 | 0.044328397 |
| Igsf6 | 0.714111159 | 0.041569611 | Serpine2 | −0.366136538 | 0.044460176 |
| Dna2 | 0.623513196 | 0.041569611 | Lrrn1 | 0.433060516 | 0.044460176 |
| Lrrc15 | −0.8413941 | 0.044485355 | | | |
| Clec2i | 0.817470583 | 0.044500889 | | | |
| Stk40 | −0.423188904 | 0.044684118 | | | |
| Slra7 | 1.311161815 | 0.044641596 | | | |
| Wnt16 | −1.105478639 | 0.044859237 | | | |
| Kcnk13 | 0.56761148 | 0.044859237 | | | |
| Fhad1 | 1.522754044 | 0.046286847 | | | |
| Svbp | 0.362532279 | 0.045902211 | | | |
| Srgap1 | −0.545932139 | 0.04597027 | | | |
| Ifi213 | 0.889138759 | 0.045995667 | | | |
| Morc3 | 0.320336656 | 0.045995667 | | | |
| Cd180 | 0.831454606 | 0.045995667 | | | |
| Trim13 | 0.441029275 | 0.045995667 | | | |
| Pianp | 0.930559587 | 0.045995667 | | | |
| Gm36161 | 0.818110366 | 0.045995667 | | | |
| Adamts16 | −0.82521823 | 0.045995667 | | | |
| Pcdh18 | −0.494062581 | 0.046190835 | | | |
| Hlf | 0.459349187 | 0.046418932 | | | |
| Amz1 | 0.680259234 | 0.04678159 | | | |
| Bcorl1 | −0.334571079 | 0.047634494 | | | |
| Arhgap15 | 0.783272879 | 0.047641588 | | | |
| Gucy1b2 | −1.428563918 | 0.047939781 | | | |
| Tmem132a | 0.323782877 | 0.047939781 | | | |
| Bex1 | 0.426685795 | 0.04799333 | | | |
| Cdc42se2 | 0.325897344 | 0.04799333 | | | |
| Smc4 | 0.501636764 | 0.048225116 | | | |
| Abcg3 | 0.716376154 | 0.048461459 | | | |
| Mir142hg | 1.124262812 | 0.048486991 | | | |
| Ifit1bl1 | 1.226810416 | 0.048811929 | | | |
| Dusp18 | −0.439769454 | 0.048811929 | | | |
| Barx2 | −0.505824196 | 0.048811929 | | | |
| Sp110 | 0.561438334 | 0.049084886 | | | |
| Plat | −0.452959728 | 0.049084886 | | | |
| Vcan | −0.567021246 | 0.049084886 | | | |
| Evi2a | 0.519206368 | 0.049237412 | | | |
| Bcar3 | 0.435503585 | 0.049855187 | | | |

[symbol]

indicates data missing or illegible when filed

A direct comparison of the transcriptional responses to SEA versus rSEA in muscle 1-week was also performed after treatment and found notable decreases among multiple inflammatory signatures after rSEA treatment, and it was confirmed that most of the type 2 transcriptional responses were alike (FIG. 7B). The comparison of the transcriptional changes influenced by SEA versus rSEA found that 18 genes were statistically significant at the False Discovery Rate (FDR)<0.05 threshold, likely due to the vast similarity between SEA and rSEA. Direct comparison found that muscles treated with rSEA led to differential expression for upregulated genes in comparison to SEA treatment, such as Angptl7, Ddit4, Klf11, and Tsc22d3 which are associated with cell proliferation, lower levels of fibrosis, muscle cell adherence, and responses to IL-10 anti-inflammation. Genes found to be downregulated in rSEA (upregulated in SEA) included Sele (E-Selectin), Cxcr2 (IL-18RB), Clec4e, and Slfn4 which are associated with reduced trafficking of inflammatory cells, and reductions in IL-17A associated allergy and fibrotic responses mediated by dendritic cell activity. For a discovery focused analysis of our direct comparison of SEA and rSEA, the FDR threshold was further adjusted and it was found that many pro-inflammatory genes were downregulated specific to rSEA treatment as the qRT-PCR assays had suggested. The largest differences in transcriptional magnitude was compared between SEA and rSEA treatment when each is referenced to the saline treated control muscles and found that rSEA trended towards lower expression of inflammatory genes such as Cxcl5, Illf9 (IL-36γ), Saa3, and Illb, higher expression of genes associated with cell proliferation such as Gsg1 and Angptl7, increases in alternatively activated macrophage polarization genes such as Retnla and Cd209e, and increases of adipose browning genes (Elovl3) (FIG. 7B).

Previous studies with ECM biomaterials demonstrated that T_{H}2 cells are critical for the pro-regenerative response to ECM biological scaffolds so the T cell response to rSEA was further examined (4, 37, 38). At 1-week post-treatment, CD3⁺ T cell numbers increased in the muscle wound with CD3⁺CD4⁺ T cells increasing over 7-fold in the muscle with rSEA treatment compared to saline treated controls. Treatment with rSEA significantly increased T_{H}2 (CD3⁺CD4⁺GFP⁺ (IL4⁺)) as a percentage of CD3⁺ cells in the muscle of 4get mice (FIG. 18G). Since the 4get mice report the transcriptional status of the IL-4 locus via GFP expression and have a BALB/c background, it was further confirmed that direct increases in IL-4 protein with intracellular cytokine staining (ICS) flow cytometry in C57BL/6 mice and found significant increases in T_{H}2 cells (CD3⁺CD4⁺IL4⁺) with rSEA treatment (FIG. 8C). There were also changes in the more inflammatory subsets of T cells with rSEA treatment including a significant decrease in the percentage of IFN-γ expressing CD4⁺ T cells, a decreased percentage of CD8⁺ T cells, and decreased percentage of IFN-γ+ natural killer and natural killer T cells (FIG. 18H) suggesting that rSEA does not induce negative inflammatory changes in addition to the pro-regenerative response.

T cell gene expression changes with rSEA treatment was further characterized by sorting CD3⁺ T cells from 1-week post-injury muscle and analyzing gene expression signatures with the NanoString multiplex system. There were 20 genes significantly upregulated and downregulated in T cells with rSEA treatment (FIG. 18I, Table 9).

TABLE 9
1-week post-injury: CD3⁺SSC^{Low} sorted cells from skeletal muscle

| | Log2 | Std | Lower | Upper | P- | | Analysis | nCounter |
|---|---|---|---|---|---|---|---|---|
| Gene | FC | Error | Conf. Limit | Cont. Limit | value | BY.p.value | Method | Probe.ID |
| Il10 | 3.49 | 0.778 | 1.97 | 5.02 | 0.00203 | 0.44 | lm.nb | NM_010548.1: 985 |
| Il10ra | 3.03 | 0.527 | 2 | 4.07 | 0.00043 | 0.33 | loglinear | NM_008348.2: 75 |
| Tnfrsf18 | 2.99 | 0.425 | 2.15 | 3.82 | 0.00011 | 0.18 | loglinear | NM_009400.2: 840 |
| Cer4 | 2.15 | 0.498 | 1.18 | 3.13 | 0.00253 | 0.47 | lm.nb | NM_009916.2: 394 |
| Il13 | 1.92 | 0.352 | 1.23 | 2.6 | 0.00061 | 0.33 | loglinear | NM_008355.2: 425 |
| Isg20 | 1.9 | 0.428 | 1.06 | 2.74 | 0.00215 | 0.44 | lm.nb | NM_020583.5: 552 |
| Nfatc3 | 1.86 | 0.383 | 1.11 | 2.63 | 0.00125 | 0.38 | loglinear | NM_010901.2: 2260 |
| Tigit | 1.65 | 0.391 | 0.885 | 2.42 | 0.00291 | 0.49 | lm.nb | NM_001146325.1: 730 |
| Cxer6 | 1.48 | 0.252 | 0.987 | 1.98 | 0.00037 | 0.33 | lm.nb | NM_030712.4: 650 |
| Cd5 | 1.46 | 0.296 | 0.883 | 2.04 | 0.00113 | 0.38 | loglinear | NM_007650.3: 1395 |
| Ly9 | 1.45 | 0.337 | 0.788 | 2.11 | 0.0026 | 0.47 | loglinear | NM_008534.2: 1190 |
| Cd4 | 1.37 | 0.188 | 1 | 1.74 | 8.63E−05 | 0.18 | loglinear | NM_013488.2: 950 |
| Cer2 | 1.17 | 0.213 | 0.751 | 1.59 | 0.00059 | 0.33 | lm.nb | NM_009915.2: 2965 |
| Guta3 | 1.17 | 0.247 | 0.686 | 1.65 | 0.00147 | 0.38 | loglinear | NM_008091.3: 1943 |
| Cd3e | 1.1 | 0.226 | 0.653 | 1.54 | 0.00128 | 0.38 | lm.nb | NM_007648.4: 380 |
| Il2rg | 1.09 | 0.263 | 0.578 | 1.61 | 0.00318 | 0.49 | loglinear | NM_013563.3: 1226 |
| H2-Au | −0.916 | 0.189 | −1.29 | −0.546 | 0.00126 | 0.38 | lm.nb | NM_010378.2: 450 |
| Cd81 | −1.89 | 0.26 | −1.6 | −0.582 | 0.003 | 0.49 | lm.nb | NM_133655.2: 575 |
| Cxcl12 | −1.27 | 0.312 | −1.88 | −0.66 | 0.00354 | 0.51 | lm.nb | NM_021704.3: 259 |
| Cd36 | −1.38 | 0.293 | −1.95 | −0.806 | 0.00152 | 0.38 | lm.nb | NM_007643.3: 1520 |

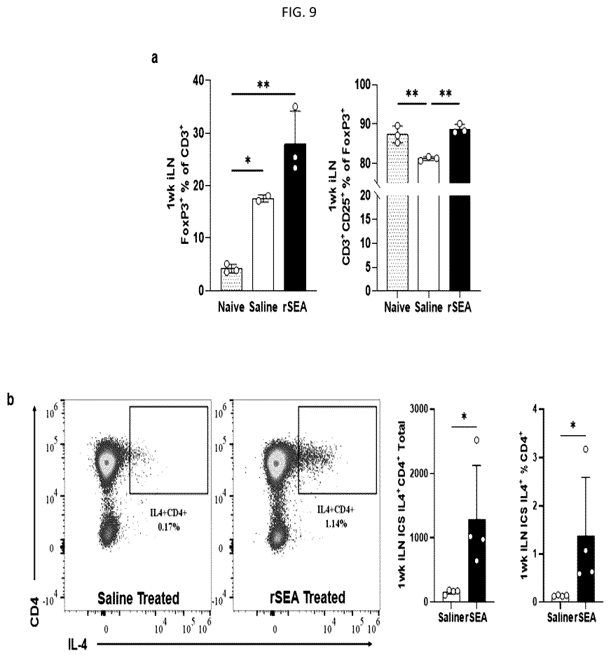

In particular, rSEA treatment of the muscle wound significantly upregulated T_{H}2 associated gene signatures including Il13, Gata3, and Cd4. Expression of Tnfrsf18, Ccr4, Il10, and Il10ra also significantly increased in the T cells after rSEA treatment suggesting a role for regulatory T cells (T_{regs}). Flow cytometry confirmed a significant increase in T_{regs} (CD3⁺CD4⁺Foxp3⁺) in the muscle with rSEA treatment compared to saline controls on days 5 and 7 post-injury (FIG. 18J), and higher T_{reg} percentages in the lymph nodes draining the muscle injury sites (FIG. 9A). As T_{regs} are known to be integral to muscle healing (39), this further supports a regenerative phenotype induced by rSEA.

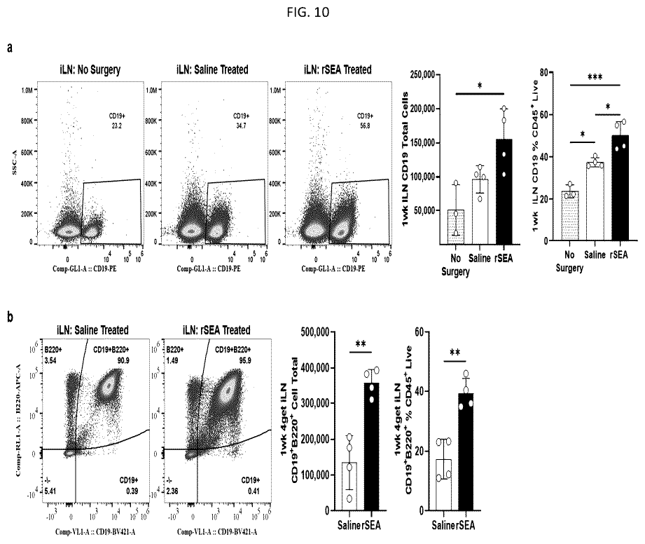

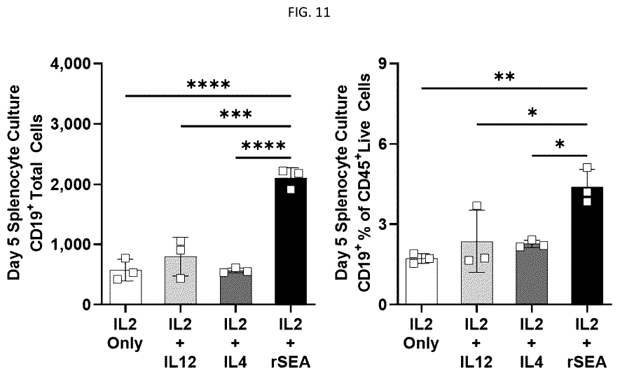

The draining inguinal lymph nodes (iLNs) also revealed type 2 immune stimulation after rSEA treatment including a significant increase in IL4⁺CD4⁺ T_{H}2 cell percentage in 4get mice (FIG. 18k) and significant increases in T_{H}2-associated gene expression signatures combined with decreased Ifng and Illb at 1-week post-injury (FIG. 18L). Similar to the muscle, confirmed of IL-4 in iLNs was further confirmed with ICS flow cytometry in C57BL/6 mice and found that rSEA induced T_{H}2 cells (CD3⁺CD4⁺IL4⁺, FIG. 9B). There was a significant decrease in the percentage of IFN-γ production in CD4⁺ T cells, a decreased percentage of CD8⁺ T cells, and decreased percentage of IFN-γ⁺ natural killer and natural killer T cells (FIG. 18M). While there were no differences in CD19⁺ B cells in the muscle 1-week post-treatment, it was found that significant changes in B cell percentages and phenotypes in the iLNs (FIG. 10A). The number of CD19⁺B220⁺ B cells in the iLNs increased compared to saline controls and the percentage of CD19⁺B220⁺ B cells also increased in the iLNs (FIG. 10B). In the in vitro splenocyte cultures, rSEA exposure significantly increased B cell numbers, suggesting a direct stimulatory role of rSEA on B cells in the regenerative immune response (FIG. 11).

rSEA Induces Alternatively Activated Macrophage Gene Expression in Muscle Wounds

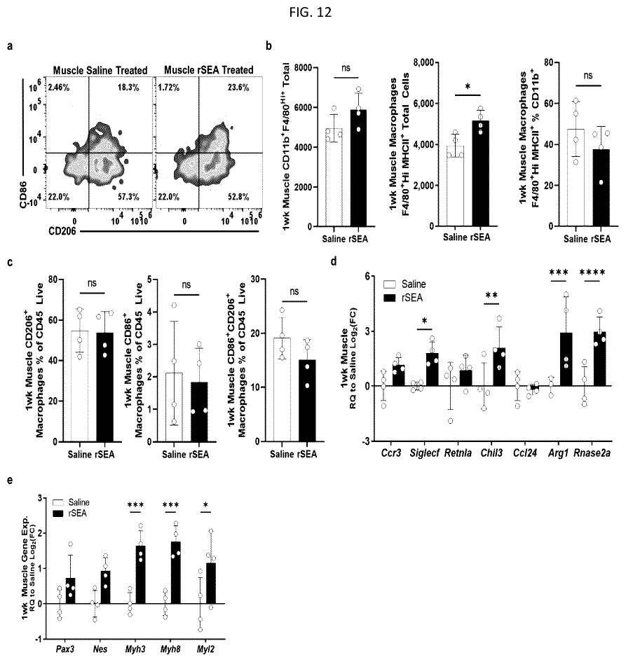

Macrophages are another immune cell type that is central to tissue repair with alternatively activated macrophages associated with productive wound healing (40). The number of macrophages in the muscle tissue had no significant change with rSEA treatment (FIG. 12A, 12B) and there were no differences in CD86 and CD206 expression (FIG. 12C). To further evaluate the macrophages, CD11b⁺F4/80^{Hi+} myeloid cells was sorted from the muscle wound 1-week post-injury and utilized the NanoString Myeloid Codeset for gene expression analysis. rSEA treatment induced significant changes in macrophage expression of 57 out of the 770 genes tested (FIG. 18N). Expression of 31 genes was found to be significantly increased with rSEA treatment related to metabolism, cell migration/recruitment, and cell activation including Chil3, Arg1, Cd163, and Ccl24 (Eotaxin-2) that are correlated with non-inflammatory macrophages (FIG. 18N). Confirming expression of these genes with qRT-PCR, it was found that rSEA treated muscle wounds significantly increased expression of Chil3, Rnase2a, and Arg1 compared to controls (FIG. 12D). The CD11b⁺F4/80^{Hi+} cells sorted from rSEA-treated muscle also significantly downregulated several genes associated with inflammation and complement activation genes such as S100a11, Itgam, Itgal, Cxcl16, and C1qc (FIG. 18N, Table 10).

TABLE 10
1-week post-injury: F4/80^{Hi+} sorted cells from skeletal muscle

| | Log2 | Std | Lower | Upper | P- | | Analysis | nCounter |
|---|---|---|---|---|---|---|---|---|
| Gene | FC | Error | Conf. Limit | Conf. Limit | value | BY.p.value | Method | Probe.ID |
| Rnuse2a | 4.43 | 0.763 | 2.94 | 5.93 | 0.000403 | 0.0974 | lm.nb | NM_053113.2: 332 |
| Chil4 | 4.42 | 0.382 | 3.67 | 5.17 | 2.80E−06 | 0.00258 | lm.nb | NM_145126.2: 545 |
| Chil3 | 4.27 | 0.288 | 3.7 | 4.83 | 4.28E−07 | 0.00118 | lm.nb | NM_009892.2: 823 |
| Arg1 | 2.39 | 0.415 | 1.58 | 3.21 | 0.000424 | 0.0974 | lm.nb | NM_007482.3: 626 |
| Csf2ra | 2.37 | 0.426 | 1.53 | 3.2 | 0.000536 | 0.114 | lm.nb | NM_009970.2: 296 |
| Lgals3 | 1.92 | 0.236 | 1.46 | 2.38 | 3.84E−05 | 0.0265 | loglinear | NM_001145953.1: 1005 |
| Siglec1 | 1.79 | 0.255 | 1.29 | 2.29 | 0.000111 | 0.051 | lm.nb | NM_011426.3: 4550 |
| Bcl2 | 1.76 | 0.589 | 0.607 | 2.91 | 0.0202 | 0.705 | Wald | NM_009741.3: 1844 |
| Ikbke | 1.64 | 0.322 | 1.01 | 2.27 | 0.000921 | 0.159 | lm.nb | NM_019777.3: 618 |
| Tlr4 | −0.82 | 0.165 | −1.14 | −0.497 | 0.00109 | 0.176 | loglinear | NM_021297.2: 2510 |
| Prdx1 | −0.835 | 0.318 | −1.46 | −0.212 | 0.034 | 0.977 | Wald | NM_011034.4: 1131 |
| Tlr2 | −0.84 | 0.273 | −1.37 | −0.305 | 0.0151 | 0.602 | lm.nb | NM_011905.2: 255 |
| S100a11 | −0.853 | 0.0681 | −0.987 | −0.72 | 1.55E−06 | 0.00213 | loglinear | NM_016740.3: 304 |
| Fegr3 | −0.863 | 0.181 | −1.22 | −0.508 | 0.00143 | 0.18 | lm.nb | NM_010188.5: 1175 |
| Cd151 | −0.896 | 0.279 | −1.44 | −0.348 | 0.0149 | 0.602 | Wald | NM_009842.3: 610 |
| Pros1 | −0.923 | 0.335 | −1.58 | −0.264 | 0.0252 | 0.827 | loglinear | NM_011173.2: 2720 |
| Itgam | −0.94 | 0.139 | −1.21 | −0.667 | 0.000143 | 0.055 | loglinear | NM_001082960.1: 3025 |
| Prdx3 | −0.958 | 0.144 | −1.24 | −0.676 | 0.00016 | 0.055 | loglinear | NM_007452.2: 496 |
| Itga1 | −1.1 | 0.234 | −1.56 | −0.642 | 0.00219 | 0.217 | Wald | NM_008400.2: 950 |
| Cysltr1 | −1.24 | 0.307 | −1.85 | −0.64 | 0.00372 | 0.244 | loglinear | NM_021476.4: 1190 |

The most strongly downregulated gene, Cysltr1, encodes for cysteinyl leukotriene receptor-1, a potent mediator of allergic inflammation. In contrast to an active helminth infection, rSEA treatment resulted in downregulation of genes such as Tlr2, Tlr4 and Jun in the sorted myeloid cells compared to saline treated controls. This suggests that the rSEA formulation can induce the regenerative components of the type 2 immune helminth response without the deleterious infection response component.

rSEA Stimulation of Type 2 Immunity Correlates with Increased Muscle Repair

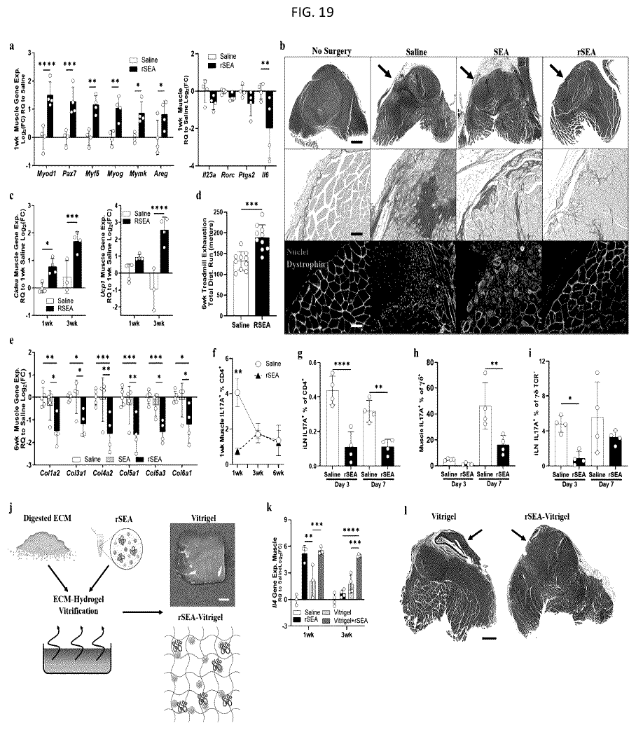

IL-4 expressing eosinophils, T_{H}2 cells and regulatory T cells are all associated with muscle repair after traumatic injury (4, 5, 39, 41). To assess whether rSEA stimulation of type 2 immunity also benefitted muscle repair, healing and fibrosis were assessed with histology and further expression analysis at early (1-week) and late (6-week) timepoints. At 1-week post-surgery time point, where it was found that broad type 2 immune stimulation, there was a significant increase in expression of genes associated with muscle satellite cell activation (Pax7, Myod1, Myf5, Myog, Mymk, and Areg) and myofiber fusion and development (Myh3, Myh8, and Myl2) with rSEA treatment compared to saline treated controls (FIG. 19A, FIG. 12E). The increased expression of muscle development genes with rSEA at early time points correlated with increased muscle tissue. At 6-weeks post-rSEA treatment, immunofluorescent staining of dystrophin, a marker of mature muscle tissue, increased with rSEA treatment compared to saline controls (FIG. 19B). Moreover, the rSEA treated muscle had centrally located nuclei, a characteristic of regenerating muscle tissue, compared to peripheral nuclei in the saline treated controls. In addition to increased muscle tissue, rSEA treatment reduced fibrosis. Masson's trichrome staining of the muscle injury showed reduced collagen deposition without granuloma formation with rSEA treatment (FIG. 19B). While the majority of upregulated type 2 associated genes had waned by 3-weeks post-injury and rSEA treatment, it was found that the expression of adipose browning genes, Ucp1 and Cidea, had strongly increased at this time range in the muscle (FIG. 19C) At 6-weeks post-injury, functional testing of the injured mice was performed using a treadmill exhaustion assay and found that rSEA treatment led to a significant increase in the running distance achieved by the mice compared to saline vehicle treatment (FIG. 19D). Gene expression of the muscle tissue at 6-weeks post-injury, supported the morphological findings with decreased expression of fibrosis-associated genes (Col1a1, Col5a1, Col6a1), decreased ratio of Col1a2 to Col3al with rSEA treatment relative to saline controls (FIG. 18E).

rSEA Treatment Decreases IL-17A Producing CD4⁺ and γδ⁺ T Cells

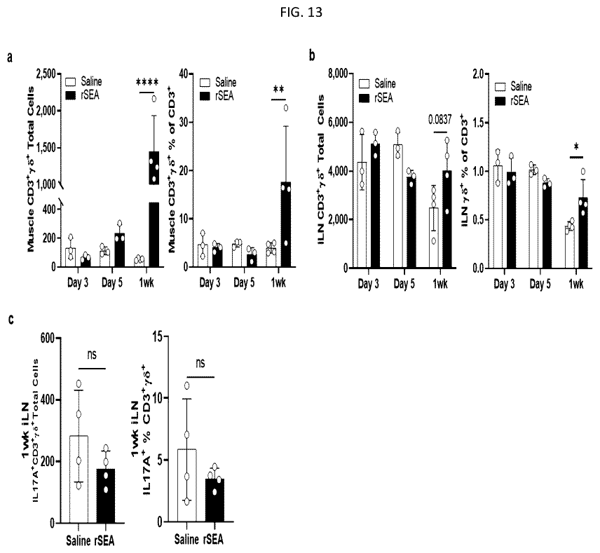

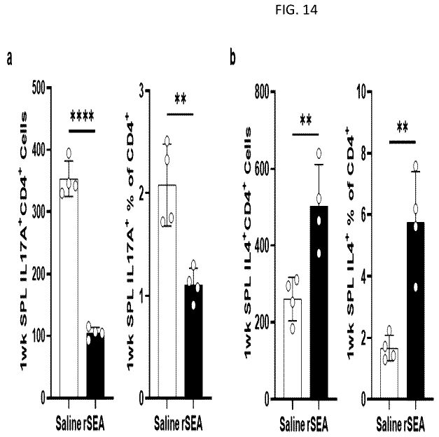

Schistosome eggs deposited in tissues can induce fibrosis over time and induce granuloma formation similar to the foreign body response. Furthermore, SEA has been used as a model for fibrosis when coated on glass, -sepharose, and -polystyrene beads in multiple tissues including liver and lung (28, 42). As fibrosis and granuloma formation are not desirable outcomes in tissue repair, it was therefore sought to further evaluate immunological features associated with fibrosis, specifically type 3 (17) immune cells (43, 44). Expression of type 3 immune-associated genes including Il23a, Rorc, Ptgs2 (COX2) and Il6 decreased in the muscle tissue 1-week after injury and rSEA treatment (FIG. 19A). IL-17A, central to type 3 immune responses, is implicated in fibrosis in multiple tissues including lung, liver, skeletal muscle, and in fibrosis associated with the foreign body response (43-46). In parallel with the reduced fibrosis it was observed that histologically that rSEA treatment significantly decreased IL-17A producing T_{H}17 cell number and percentages in the muscle at day 7 post-injury compared to saline, returning to similar levels at 3 and 6 weeks (FIG. 19f). Further exploring the earlier time points, rSEA induced the most significant decrease in T_{H}17 cells at 3 days with a significant decrease still present at 7 days post treatment compared to saline (FIG. 19G). rSEA treatment also impacted IL-17A expression by γδ T cells (FIG. 19H, FIG. 19I). While γδ T cell numbers significantly increased after rSEA treatment at 1-week (FIG. 13), expression of IL-17A⁺γδ⁺ T cells was minimal at 3 days in both groups and significantly decreased with rSEA treatment at 7 days compared to saline treated controls (FIG. 19H, FIG. 19I). In the draining inguinal lymph node, IL-17A⁺γδ⁺ T cell percentage significantly decreased at 3 days with rSEA treatment and moderately decreased at 7 days compared to saline control (FIG. 19I, FIG. 13). A significant reduction in the number of IL17A⁺CD4⁺ cells (and percentage of CD4 and CD45) of rSEA-treated mice at 1-week post-injury was also found, suggesting that decreased inflammatory signatures are occurring more broadly in regionally and systemically in secondary lymphoid tissues (FIG. 14).

Release of rSEA from a Vitrified ECM Hydrogel Enhances the Type 2 Regenerative Response

Larger tissue defects may require a scaffold to enable cell migration and the repair of larger tissue volumes. Biological scaffolds derived from tissue ECM are used clinically for wound healing and reconstruction applications. Preclinical and clinical studies demonstrate that ECM materials can induce type 2 immune responses and promote tissue repair (37, 47, 48). Although the effects of a single dose of rSEA persist for weeks, the ability to incorporate rSEA into a biomaterial would provide a scaffold for larger tissue defects and deliver the immunotherapy over an extended period of time.

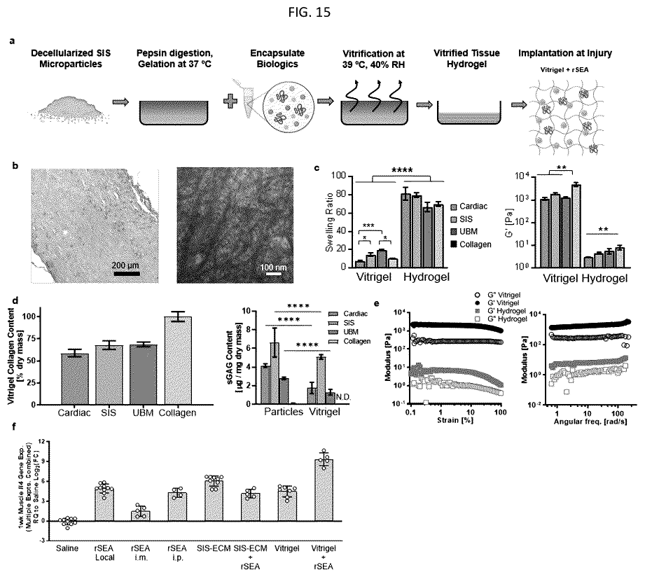

Biological scaffolds based on ECM can be processed into several material forms without compromising their regenerative capacity. Clinically formulations include powders, sheets, and hydrogels (49). Drugs can also be encapsulated into ECM hydrogels, although these gels are weak and quickly dissolve (38). To create more robust ECM hydrogels that can release proteins and lipids in a controlled manner, a vitrification process was applied that evaporates water in a controlled humidity and temperature so that the macromolecular assembly can occur while a drug or biologic is encapsulated (FIG. 19J, FIG. 15). Similar to our previous studies with vitrified collagen, vitrification of urinary bladder-derived ECM produced gels with increased matrix assembly and fiber formation, as visualized by gross histology and transmission electron microscopy (TEM) (FIG. 15B) (50, 51). The ECM hydrogel water content decreased after vitrification in parallel with an increase in mechanical properties (G′ modulus) further confirming ECM assembly and formation of a stronger material (FIG. 15C). Release profile of a model small molecule drug from the vitrified ECM confirmed controlled release over 7 days (FIG. 15D). Application of ECM particles or vitrigel (without rSEA) induced similar type 2 immune skewing (FIG. 15F).

The complex protein and lipid mixture of rSEA makes controlled release difficult to evaluate. Therefore, it was sought to evaluate rSEA-encapsulated vitrigels functionally by assessing their impact on wound healing and immune profile skewing. Particles of vitrified ECM, with or without encapsulated rSEA, were applied to the murine VML. A single dose of rSEA and rSEA-encapsulated vitrigel stimulated similar levels of Il4 in the muscle tissue at one week, however only the vitrigel+rSEA extended the increased Il4 expression to the 3-week time point compared to a single dose of rSEA, with a 30-fold increase in Il4 expression (FIG. 19K). The vitrified ECM hydrogel alone stimulated Il4 gene expression to a small degree. Histological analysis further supported enhanced muscle healing with vitrigel+rSEA treatment without accompanying fibrosis (FIG. 19L). The vitrigel ECM provided a scaffold for tissue growth in addition to controlled release of the rSEA that resulted in a larger volume of new muscle tissue. Vitrigels without rSEA are still visible in the muscle after 6-weeks compared to vitrigels with rSEA that were largely degraded, likely due to increased repair. Moreover, the overall volume of new muscle tissue was smaller in the vitrigel alone group.

rSEA Promotes Healing in Articular Cartilage and Cornea Tissue Injury Models

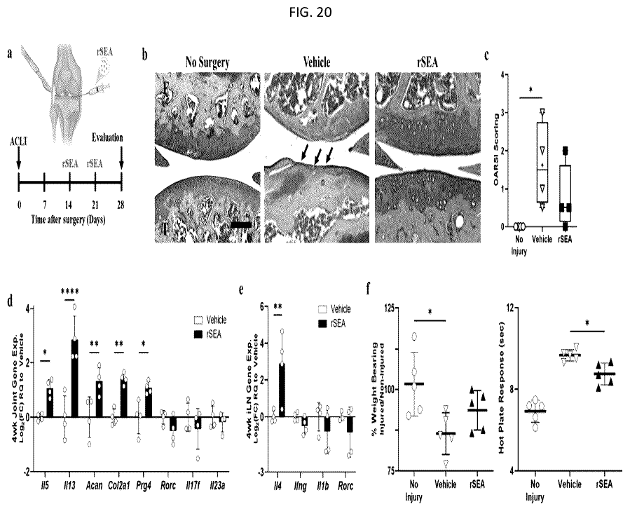

A type 2 immune response and IL-4 expression is associated with repair in multiple tissues beyond muscle including liver, articular cartilage, the central nervous system, and skin (21, 26, 41, 52-54). To determine if rSEA could be broadly applied to promote regeneration in tissues beyond muscle, the therapeutic potential of rSEA in cartilage and cornea injury models was evaluated in male mice. For cartilage repair, the anterior cruciate ligament transection (ACLT) model was used that induces articular damage, loss of cartilage and development of osteoarthritis (55). rSEA intra-articular (IA) was injected two and three weeks after the ACLT injury (FIG. 20A) and evaluated the joints 4 weeks after injury compared to vehicle injections.

Histological assessment of the articular joint structure and cartilage using Safranin-O staining for proteoglycans found that rSEA resulted in higher proteoglycan staining in the cartilage layer, improved tissue structure, and trended in higher levels of repair quality as measured by the semi-quantitative OARSI scoring system compared to vehicle controls (FIG. 20C). Gene expression analysis of the whole joint tissue at the 4-week time point supported a type 2 immune skewing. Expression of type 2 genes (Il5 and Il13) and cartilage repair and extracellular matrix markers (Prg4, Acan1, and Col2a1) increased compared to vehicle controls (FIG. 20D). Similar to the muscle injury after rSEA treatment, it was found that immune type 1 and type 17 gene expression signatures (Rorc, Il17f, and Il23a) trended downward in comparison to vehicle controls (FIG. 20D, FIG. 20E). To evaluate functional repair, nociception and weight bearing was tested of the injured limb. Hotplate nociception measures the latency period of hindlimb response to heat-induced pain, wherein shorter time responses indicate lower inflammation and more repair (55). It was found that rSEA treatment significantly decreased the nociceptive response time of the mice in comparison to vehicle treatment suggesting reduced pain (FIG. 20F). Functional weight bearing analysis, a measure of the percentage of weight placed upon the injured limb relative to the uninjured limb, improved with rSEA treatment (Vehicle: 86.44%, rSEA: 93.61%) (FIG. 20F).

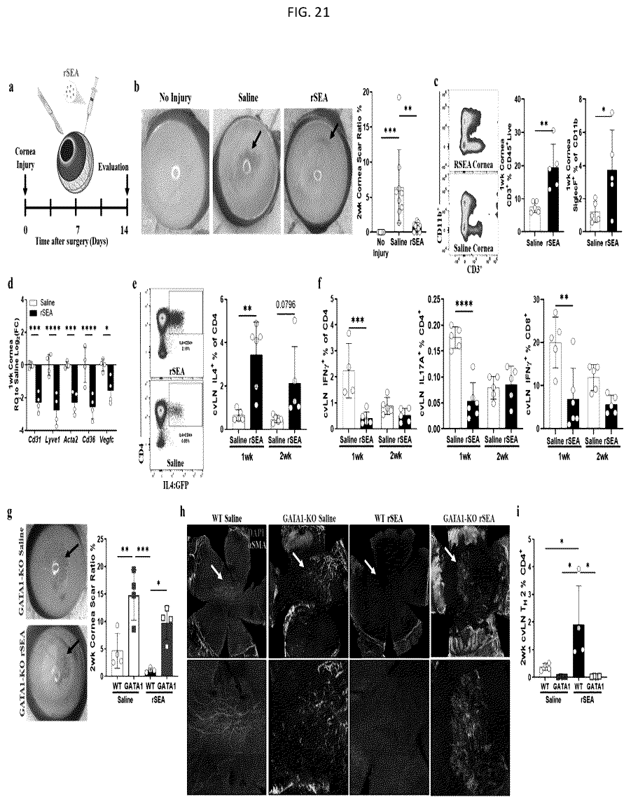

The therapeutic potential of rSEA in a cornea wound that is similarly characterized by poor healing capacity and scar formation when damaged was evaluated. In the corneal debridement injury model, resident stromal cells are activated leading to fibrosis and scarring which results in limited vision (FIG. 21A) (56, 57). rSEA was injected in the subconjunctival space immediately after injury and compared the tissue response to control saline injections. Gross imaging of the corneal surface and tissue clarity 2-weeks after injury showed a significant increase in cornea repair with rSEA treatment compared to controls as measured by blinded quantitative analysis of the scar area (FIG. 21B).

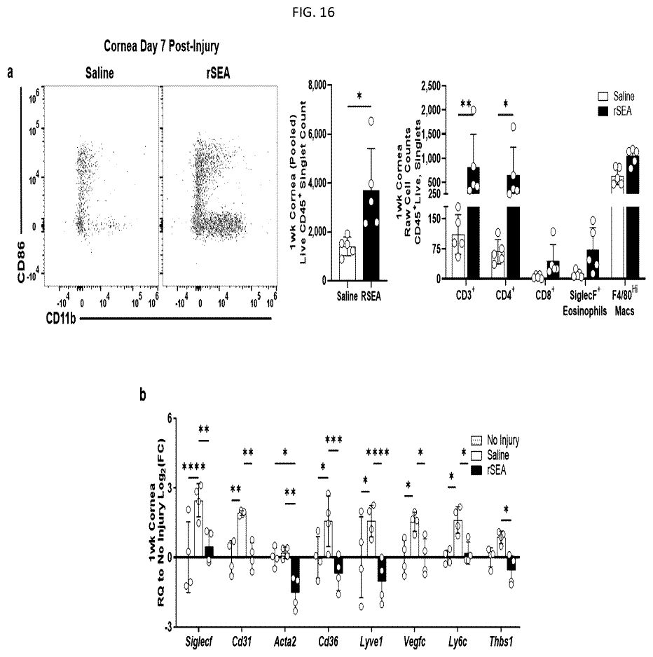

The immune response in the cornea was then examined after wounding and rSEA treatment to see if an increased type 2 profile correlated with increased tissue repair similar to the muscle and cartilage. A few changes were observed in the immune cell populations in the cornea tissue as measured by flow cytometry which may be due to the small cell numbers even when multiple corneas are combined (FIG. 21C, FIG. 16). The gene expression of the injured and rSEA-treated corneas was assessed and found a significant decrease in inflammatory and angiogenic associated genes 1-week post-treatment including Lyve1, Cd31, Vegfc, Cd36, and Acta2 (FIG. 21D, FIG. 16B). In the draining cervical lymph nodes however, there was a significant increase in IL-4-expressing CD4⁺ T cells one week after rSEA treatment and moderate increase at 2 weeks (FIG. 4e). In parallel, there was a significant decrease in IL-17A⁺CD4⁺, IFN-γ+CD4⁺, IFN-γ+CD8⁺ T cells percentage with rSEA treatment 1-week post-treatment with minimal changes at 2 weeks (FIG. 21F).

Since rSEA treatment increased eosinophil migration in other tissues it was hypothesized that eosinophils may be contributing to rSEA-mediated cornea repair. In the Δdb1GATA model that does not have eosinophils, the scar area significantly increased in size with or without rSEA treatment (FIG. 21G). Furthermore, the αSMA immunofluorescence staining decreased with rSEA treatment in wild-type mice, but it was found that this increased with or without treatment in eosinophil-deficient mice (Δdb1GATA), suggesting impaired wound healing and increased fibrosis (FIG. 21H). Immunological analysis of the draining cervical lymph node revealed that the significant increase in T_{H}2 cells induced by rSEA in WT animals is completely ablated in Δdb1GATA mice (FIG. 21I). This finding contrasts with the VML model (FIG. 21E) and thus suggests that eosinophils are important effectors in healing of the cornea wound by rSEA-induced T_{H}2 responses. Thus, rSEA treatment acts on multiple cell types, whose importance in the regenerative process depends on the tissue type and wound.

In this study, a pro-regenerative immunotherapy derived from fractionated helminth parasite egg antigens was designed and its ability to enhance wound healing and deter fibrosis post-traumatic injury across three injury models demonstrated. Taking the eggs from S. mansoni helminths, an alternative formulation from the soluble egg antigen, rSEA was derived. It was shown that the rSEA stimulated a type 2 immune signature in lymphoid cells and myeloid cells, further decreasing pro-inflammatory immune polarization, and later timepoints revealed decreased levels of fibrosis associated with inhibition of T_{H}17 and γδ⁺IL-17A⁺ cells. Application of rSEA to muscle, cornea, and articular joint injuries generally improved tissue healing assessed by gene expression signatures, cell populations, and/or histological assessment. Controlled release of rSEA from a natural sourced and decellularized biomaterial hydrogel further promoted healing and regeneration of larger tissue volumes. The rSEA formulation described herein, particularly in the form of an ECM hydrogel, is therefore a regenerative immunotherapy with potentially broad application to tissue repair and homeostasis, though several vital questions remain for exploration in future work such as the optimal formulations and scaling of SEA to benefit healing, deleterious off-target effects, and immunotherapy-induced susceptibility to other pathogens during treatment.

### Discussion

A type 2 immune response is central to how the immune system responds to helminth infection. While the type 2 response has long been considered an anti-helminth response, it is also now believed that helminths may induce this anti-inflammatory immune signature to repair the damage caused to the host, thereby enhancing mutual survival. Recent studies highlight the importance of the context of expression of type 2 associated molecules that are important in dictating outcomes, such as amphiregulin, IL-13 and IL-33 (20). IL-4 and type 2 immunity is associated with tissue repair and healing in multiple tissue types including liver (52), bone (53), cartilage (48), muscle (4, 5), corneal (57), and nervous tissues (26, 54), suggesting that rSEA may be broadly applicable for tissue repair (18, 20, 21). Treatment with rSEA induced an immune profile that included eosinophils and T_{H}2 cells producing significantly higher levels of IL-4, IL-5, and IL-13 protein or gene expression compared to injured tissue without treatment. The type of tissue where injury and treatment may impact which cells are responding to rSEA and promoting tissue repair. In these studies, application of rSEA to a cornea wound in the Δdb1GATA murine cornea injury model completely abolished repair. However, in skeletal muscle injuries the Δdb1GATA mice did not ablate pro-healing gene expression signatures induced by rSEA despite their well-recognized role in muscle tissue repair (5). This suggests that rSEA may activate multiple immune cell populations to promote repair that differ according to tissue type. It is also likely that rSEA influences stromal, stem, or progenitor cell populations in addition to immune cells as Helminth infections were shown to stimulate stem cells in the intestinal niche (19).

Sex differences in immune responses contribute to autoimmune disease, infection and vaccine responses. While sex differences in the immune response to tissue damage and contributions to repair are likely, these differences are not well-studied and currently remain largely unknown. In these studies, the rSEA response in a muscle injury in female mice and the articular and cornea injuries in male mice was tested so they could be compared to previous work in the respective fields (58-61). The rSEA treatment improved tissue repair in both male and female mice in their specific injuries.

It is understood that the foregoing detailed description and accompanying examples are merely illustrative and are not to be taken as limitations upon the scope of the disclosure, which is defined solely by the appended claims and their equivalents.

Various changes and modifications to the disclosed aspects will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### REFERENCES

1. T. E. G. Krueger, D. L. J. Thorek, S. R. Denmeade, J. T. Isaacs, W. N. Brennen, Concise Review: Mesenchymal Stem Cell-Based Drug Delivery: The Good, the Bad, the Ugly, and the Promise. Stem Cells Transl Med 7, 651-663 (2018).
2. C. Chabannon et al., Hematopoietic stem cell transplantation in its 60s: A platform for cellular therapies. Sci Transl Med 10, (2018).
3. P. Laurent et al., Immune-Mediated Repair: A Matter of Plasticity. Front Immunol 8, 454 (2017).
4. K. Sadtler et al., Developing a pro-regenerative biomaterial scaffold microenvironment requires T helper 2 cells. Science 352, 366-370 (2016).
5. J. E. Heredia et al., Type 2 innate signals stimulate fibro/adipogenic progenitors to facilitate muscle regeneration. Cell 153, 376-388 (2013).
6. D. G. Colley, A. L. Bustinduy, W. E. Secor, C. H. King, Human schistosomiasis. The Lancet 383, 2253-2264 (2014).
7. D. P. McManus et al., in Seminars in immunopathology. (Springer, 2020), vol. 42, pp. 355-371.
8. T. Vos et al., Years lived with disability (YLDs) for 1160 sequelae of 289 diseases and injuries 1990-2010: a systematic analysis for the Global Burden of Disease Study 2010. Lancet 380, 2163-2196 (2012).
9. M. L. Burke et al., Immunopathogenesis of human schistosomiasis. Parasite Immunol 31, 163-176 (2009).
10. B. B. Graham, A. P. Bandeira, N. W. Morrell, G. Butrous, R. M. Tuder, Schistosomiasis-associated pulmonary hypertension: pulmonary vascular disease: the global perspective. Chest 137, 20S-29S (2010).
11. B. J. Vennervald et al., Detailed clinical and ultrasound examination of children and adolescents in a Schistosoma mansoni endemic area in Kenya: hepatosplenic disease in the absence of portal fibrosis. Trop Med Int Health 9, 461-470 (2004).
12. K. S. Warren, A. A. Mahmoud, P. Cummings, D. J. Murphy, H. B. Houser, Schistosomiasis mansoni in Yemeni in California: duration of infection, presence of disease, therapeutic management. Am J Trop Med Hyg 23, 902-909 (1974).
13. T. P. Brosschot, L. A. Reynolds, The impact of a helminth-modified microbiome on host immunity. Mucosal Immunol 11, 1039-1046 (2018).
14. T. P. Jenkins et al., Schistosoma mansoni infection is associated with quantitative and qualitative modifications of the mammalian intestinal microbiota. Scientific reports 8, 1-10 (2018).
15. R. M. Maizels, Regulation of immunity and allergy by helminth parasites. Allergy 75, 524-534 (2020).
16. C. W. Su et al., Helminth-Induced and Th2-Dependent Alterations of the Gut Microbiota Attenuate Obesity Caused by High-Fat Diet. Cell Mol Gastroenterol Hepatol 10, 763-778 (2020).
17. M. M. Zaiss, N. L. Harris, Interactions between the intestinal microbiome and helminth parasites. Parasite immunology 38, 5-11 (2016).
18. W. C. Gause, T. A. Wynn, J. E. Allen, Type 2 immunity and wound healing:
   evolutionary refinement of adaptive immunity by helminths. Nat Rev Immunol 13, 607-614 (2013).
19. Y. M. Nusse et al., Parasitic helminths induce fetal-like reversion in the intestinal stem cell niche. Nature 559, 109-113 (2018).
20. F. Chen et al., An essential role for T_{H} 2-type responses in limiting acute tissue damage during experimental helminth infection. Nature medicine 18, 260-266 (2012).
21. R. L. Gieseck III, M. S. Wilson, T. A. Wynn, Type 2 immunity in tissue repair and fibrosis. Nature Reviews Immunology 18, 62 (2018).
22. B. Everts et al., Omega-1, a glycoprotein secreted by Schistosoma mansoni eggs, drives Th2 responses. J Exp Med 206, 1673-1680 (2009).
23. M. Okano, A. R. Satoskar, K. Nishizaki, D. A. Harn, Lacto-N-fucopentaose III found on Schitosoma mansoni egg antigens functions as adjuvant for proteins by inducing Th2-type response. The Journal of Immunology 167, 442-450 (2001).
24. G. Schramm et al., Cutting edge: IPSE/alpha-1, a glycoprotein from Schistosoma mansoni eggs, induces IgE-dependent, antigen-independent IL-4 production by murine basophils in vivo. J Immunol 178, 6023-6027 (2007).
25. T. Kiyota et al., CNS expression of anti-inflammatory cytokine interleukin-4 attenuates Alzheimer's disease-like pathogenesis in APP+PS1 bigenic mice. FASEB J 24, 3093-3102 (2010).
26. X. Liu et al., Interleukin-4 is essential for microglia/macrophage M2 polarization and long-term recovery after cerebral ischemia. Stroke 47, 498-504 (2016).
27. A. Quarta, Z. Berneman, P. Ponsaerts, Neuroprotective modulation of microglia effector functions following priming with interleukin 4 and 13: Current limitations in understanding their mode-of-action. Brain, behavior, and immunity, (2020).
28. D. L. Boros, K. S. Warren, Delayed hypersensitivity-type granuloma formation and dermal reaction induced and elicited by a soluble factor isolated from Schistosoma mansoni eggs. J Exp Med 132, 488-507 (1970).
29. D. Ditgen et al., Harnessing the helminth secretome for therapeutic immunomodulators. Biomed Res Int 2014, 964350 (2014).
30. M. M. Harnett, W. Harnett, Can parasitic worms cure the modern world's ills? Trends in parasitology 33, 694-705 (2017).
31. M. Giera et al., The Schistosoma mansoni lipidome: Leads for immunomodulation. Anal Chim Acta 1037, 107-118 (2018).
32. J. Jang-Lee et al., Glycomics analysis of Schistosoma mansoni egg and cercarial secretions. Mol Cell Proteomics 6, 1485-1499 (2007).
33. W. Mathieson, R. A. Wilson, A comparative proteomic study of the undeveloped and developed Schistosoma mansoni egg and its contents: the miracidium, hatch fluid and secretions. Int J Parasitol 40, 617-628 (2010).
34. C. L. Cass et al., Proteomic analysis of Schistosoma mansoni egg secretions. Mol Biochem Parasitol 155, 84-93 (2007).
35. M. C. Basil, B. D. Levy, Specialized pro-resolving mediators: endogenous regulators of infection and inflammation. Nat Rev Immunol 16, 51-67 (2016).
36. J. F. Markworth et al., Resolvin D1 supports skeletal myofiber regeneration via actions on myeloid and muscle stem cells. JCI Insight 5, (2020).
37. K. C. Sasse, D. L. Warner, D. Ackerman, J. Brandt, Parastomal hernia repair with urinary bladder matrix grafts: a case series. Int. J. Case Rep. Images 7, 85-91 (2016).
38. R. M. Wang, K. L. Christman, Decellularized myocardial matrix hydrogels: In basic research and preclinical studies. Adv Drug Deliv Rev 96, 77-82 (2016).
39. D. Burzyn et al., A special population of regulatory T cells potentiates muscle repair. Cell 155, 1282-1295 (2013).
40. D. M. Mosser, J. P. Edwards, Exploring the full spectrum of macrophage activation. Nat Rev Immunol 8, 958-969 (2008).
41. V. Salmon-Ehr et al., Implication of interleukin-4 in wound healing. Lab Invest 80, 1337-1343 (2000).
42. K. K. Takaki, G. Rinaldi, M. Berriman, A. J. Pagan, L. Ramakrishnan, Schistosoma mansoni Eggs Modulate the Timing of Granuloma Formation to Promote Transmission. Cell Host Microbe 29, 58-67 e55 (2021).
43. L. Chung et al., Interleukin 17 and senescent cells regulate the foreign body response to synthetic material implants in mice and humans. Sci Transl Med 12, (2020).
44. T. Fabre et al., Type 3 cytokines IL-17A and IL-22 drive TGF-beta-dependent liver fibrosis. Sci Immunol 3, (2018).
45. M. G. Shainheit et al., The pathogenic Th17 cell response to major schistosome egg antigen is sequentially dependent on IL-23 and IL-1β. The Journal of Immunology 187, 5328-5335 (2011).
46. K. Ramani, P. S. Biswas, Interleukin-17: Friend or foe in organ fibrosis. Cytokine 120, 282-288 (2019).
47. J. Dziki et al., An acellular biologic scaffold treatment for volumetric muscle loss: results of a 13-patient cohort study. NPJ Regen Med 1, 16008 (2016).
48. P. Bettiol, C. Cox, C. Gerzina, J. Simpson, B. MacKay, Novel Use of a Porcine Bladder Extracellular Matrix Scaffold to Treat Postoperative Seroma in a Total Knee Arthroplasty Patient. Arthroplasty today 7, 143-147 (2021).
49. M. T. Spang, K. L. Christman, Extracellular matrix hydrogel therapies: In vivo applications and development. Acta Biomaterialia 68, 1-14 (2018).
50. X. Wang et al., Multifunctional synthetic Bowman's membrane-stromal biomimetic for corneal reconstruction. Biomaterials 241, 119880 (2020).
51. X. Wang et al., Collagen vitrigels with low-fibril density enhance human embryonic stem cell-derived retinal pigment epithelial cell maturation. Journal of Tissue Engineering and Regenerative Medicine 12, 821-829 (2018).
52. Y. P. Goh et al., Eosinophils secrete IL-4 to facilitate liver regeneration. Proc Natl Acad Sci USA 110, 9914-9919 (2013).
53. L. Gong et al., An interleukin-4-loaded bi-layer 3D printed scaffold promotes osteochondral regeneration. Acta Biomater 117, 246-260 (2020).
54. J. Yang et al., Interleukin-4 ameliorates the functional recovery of intracerebral hemorrhage through the alternative activation of microglia/macrophage. Frontiers in neuroscience 10, 61 (2016).
55. M. Z. Ruan, R. M. Patel, B. C. Dawson, M. M. Jiang, B. H. Lee, Pain, motor and gait assessment of murine osteoarthritis in a cruciate ligament transection model. Osteoarthritis Cartilage 21, 1355-1364 (2013).
56. M. A. Stepp et al., Wounding the cornea to learn how it heals. Experimental eye research 121, 178-193 (2014).
57. X. Wang et al., Type 2 immunity induced by bladder extracellular matrix enhances corneal wound healing. Sci Adv 7, eabe2635 (2021).
58. B. A. Christiansen et al., Non-invasive mouse models of post-traumatic osteoarthritis. Osteoarthritis and cartilage 23, 1627-1638 (2015).
59. S. M. Greising, B. T. Corona, C. McGann, J. K. Frankum, G. L. Warren, Therapeutic approaches for volumetric muscle loss injury: a systematic review and meta-analysis. Tissue Engineering Part B: Reviews 25, 510-525 (2019).
60. T. Histing et al., Small animal bone healing models: standards, tips, and pitfalls results of a consensus meeting. Bone 49, 591-599 (2011).
61. S. L. Klein, K. L. Flanagan, Sex differences in immune responses. Nature Reviews Immunology 16, 626-638 (2016).
62. C. N. Serhan, B. D. Levy, Resolvins in inflammation: emergence of the pro-resolving superfamily of mediators. The Journal of clinical investigation 128, 2657-2669 (2018).
63. S. Frahm et al., A novel cell-free method to culture Schistosoma mansoni from cercariae to juvenile worm stages for in vitro drug testing. PLOS Negl Trop Dis 13, e0006590 (2019).
64. M. Z. Ruan et al., Proteoglycan 4 expression protects against the development of osteoarthritis. Sci Transl Med 5, 176ra134 (2013).
65. S. S. Glasson, M. G. Chambers, W. B. Van Den Berg, C. B. Little, The OARSI histopathology initiative—recommendations for histological assessments of osteoarthritis in the mouse. Osteoarthritis Cartilage 18 Suppl 3, S17-23 (2010).
66. T. J. Keane, I. T. Swinehart, S. F. Badylak, Methods of tissue decellularization used for preparation of biologic scaffolds and in vivo relevance. Methods 84, 25-34 (2015).
67. K. J. Livak, T. D. Schmittgen, Analysis of relative gene expression data using real-time quantitative PCR and the 2−ΔΔCT method, methods 25, 402-408 (2001).
68. M. Ogawa et al., Eosinophils promote corneal wound healing via the 12/15-lipoxygenase pathway. FASEB J 34, 12492-12501 (2020).
69. H. J. Faust et al., IL-17 and immunologically induced senescence regulate response to injury in osteoarthritis. J Clin Invest 130, 5493-5507 (2020).

1. A helminth regenerative composition comprising a mixture of a soluble fraction and an insoluble fraction, wherein the composition is produced by a process comprising the steps of:
homogenizing helminth eggs;
centrifuging the homogenized helminth eggs to form an insoluble fraction, a soluble fraction, and an egg shell fraction;
removing the egg shell layer fraction;
ultra centrifuging the soluble fraction and insoluble fraction;
filtering each of the soluble and insoluble layers separately; and
combining the filtered soluble fraction with the filtered insoluble fraction in a ratio sufficient to reduce a proinflammatory response when administered to a subject, wherein the regenerative composition comprises a mixture of the soluble fraction and the insoluble fraction.

2. The composition of claim 1, wherein the helminth is Schistosoma mansoni.

3. The composition of claim 1, wherein the soluble fraction comprises at least one helminth egg antigen.

4. The composition of claim 3, wherein the at least one helminth egg antigen is one or more of omega-1, IPSE/alpha-1, secretory glycoprotein kappa-5, smp40, Histone H2A, fructose-bisphosphate aldolase, lacto-N-fucopentaose-III (LNFPIII), lacto-n-Neotetraose (LNnT), or any combinations thereof.

5. The composition of claim 1, wherein the insoluble fraction comprises at least one lipid.

6. The composition of claim 5, wherein the at least one lipid is one or more of prostaglandin D2 (PGD2), prostaglandin E2 (PGE2), 5-hydroxyeicosatetraenoic (HETE), 15-HETE, linoleic acid, arachidonic acid, docosahexaenoic acid, 5S,18R-dihydroxy-6E,8Z,11Z,14Z,16E-eicosapentaenoic acid, 10S,17S-dihydroxydocosa-4Z,7Z,11E,13Z,15E,19Z-hexaenoic acid, (4Z,7Z,10R,11E,13E,15Z,17S,19Z)-10,17-dihydroxydocosa-4,7,11,13,15,19-hexaenoic acid, lysophosphatidylcholine, or any combinations thereof.

7. The composition of claim 1, wherein the helminthi eggs are at least about 90% homogenized.

8. The composition of claim 1, wherein the composition further comprises an extracellular matrix.

9. The composition of claim 1, wherein the composition is in incorporated into a gel or hydrogel.

10. The composition of claim 9, wherein the composition is a vitrified gel.

11. The composition of claim 10, wherein the composition, when administered to a subject, increases IL-4 and decreases IL-17.

12. A method of treating a wound or injury to a tissue in a subject, the method comprising the step of administering to a subject in need of treatment thereof, an effective amount of the composition of claim 1.

13. The method of claim 12, wherein the subject is a human.

14. The method of claim 12, wherein the tissue is muscle, an organ, cartilage, a ligament, skin, bone, nervous tissue, corneal tissue, a lens, or any combination thereof.

15. A kit comprising the composition of claim 1.
